(19) United States
(12) Patent Application Publication
(10) Pub. No.: US 2017/0079966 A1
(43) Pub. Date: Mar. 23, 2017

(54) INHIBITION OF TUMOR CELL INTERACTIONS WITH THE MICROENVIRONMENT RESULTING IN A REDUCTION IN TUMOR GROWTH AND DISEASE PROGRESSION

(71) Applicant: Deciphera Pharmaceuticals, LLC, Lawrence, KS (US)

(72) Inventors: Daniel L. FLYNN, Lawrence, KS (US); Michael D. KAUFMAN, Lawrence, KS (US); Bryan D. SMITH, Lawrence, KS (US)

(21) Appl. No.: 14/883,109

(22) Filed: Oct. 14, 2015

Related U.S. Application Data
(60) Provisional application No. 62/063,656, filed on Oct. 14, 2014.

(51) Int. Cl. A61K 31/44; A61K 9/00; A61K 39/395; C07K 16/22; A61K 45/06; C07K 16/28

(52) U.S. Cl. CPC A61K 31/44; A61K 45/06; C07K 16/2818; A61K 39/3955; C07K 16/22; A61K 9/0053; A61K 2039/505

(57) ABSTRACT

It has been shown that Compound 1 unexpectedly and potently inhibits TIE2 kinase, and that Compound 1 inhibits drug resistance mechanisms in both the tumor and in the surrounding microenvironment through balanced inhibition of TIE2, MET, and VEGFR2 kinases. Thus, Compound 1 provides a single therapeutic agent able to address multiple hallmarks of cancer by inhibiting TIE2, MET, and VEGFR2 kinases in the tumor microenvironment [Hanahan 2011]. Through its balanced inhibitory potency vs TIE2, MET, and VEGFR2, Compound 1 provides an agent which inhibits three major tumor (re)vascularization and resistance pathways (ANG, HGF, VEGF) and blocks tumor invasion and metastasis. Compound 1 exhibits anti-tumor activity alone and in combination with other targeted agents or chemotherapy.

### CROSS REFERENCE TO RELATED APPLICATIONS

This application claims priority to U.S. Provisional Application No. 62/603,656, filed Oct. 14, 2014, the contents of which are incorporated herein by reference in their entireties.

### DESCRIPTION OF THE TEXT FILE SUBMITTED ELECTRONICALLY

The content of the text file submitted electronically herewith is incorporated herein by reference in its entirety: A computer readable format copy of the Sequence Listing (filename: DECP_068_01US_SeqList_ST25.txt; date recorded Oct. 12, 2015: file size 9 KB).

### BACKGROUND

Increasingly, cancer is recognized as a complex process involving not only tumor cell specific mechanisms of transformation, but also involving cell types within the surrounding tumor microenvironment. Referred to as the hallmarks of cancer, this holistic approach to our understanding of cancer identifies cross-talk mechanisms between tumor cells and cells of the microenvironment as being essential for tumor growth, invasion, and metastasis. Critical hallmarks of cancer include 1) sustained proliferative signaling; 2) cell death resistance; 3) tumor angiogenesis; 4) invasion and metastasis; 5) inflammation; and 6) immune system avoidance [Hanahan 2011]. Cell types within the microenvironment associated with these hallmarks of cancer include vascular endothelial cells, lymphatic endothelial cells, fibroblasts, and tumor-tolerant macrophages and lymphocytes. New targeted therapeutics which block multiple hallmark mechanisms of cancer are highly sought.

The angiopoietin (ANG)/TIE2 signaling axis on endothelial cells and pro-angiogenic macrophages contributes to tumor vascularization and can mediate angiogenic signaling after anti-VEGF therapy. Such ANG/TIE2 mediated tumor vascularization has been demonstrated in breast cancer, pancreatic cancer, glioblastoma, and ovarian cancer [Mazzieri 2011; Rigamonti 2014; Schulz 1011; Brunckhorst 2010; Hata 2002]. Moreover, combination treatment with anti-VEGF and anti-ANG2 therapy leads to a more substantial and durable reduction in tumor growth in preclinical models [Hashizume 2010].

Another mechanism of tumor resistance or recurrence after anti-VEGF therapy has been attributed to tumor-infiltrating myeloid cells in response to cell death and hypoxia after vascular regression [Bergers 2008]. Tumor resistance to chemotherapy, radiotherapy or hormonal therapy has also been shown to be caused by therapy-induced recruitment of infiltrating macrophages [DeNardo 2011; Escamilla 2015]. Tumor-infiltrating macrophages are a source of cytokines and chemokines to support tumor growth, survival, tumor cell motility, avoid immune destruction, and promote angiogenesis and metastasis [Wyckoff 2004; Wyckoff, 2007; De Palma 2005; Coffelt 2011; Lin 2006].

So-called tumor-associated macrophages (TAMs) not only promote tumor growth but can limit the efficacy of the tumor response to chemotherapy [De Nardo 2011; Escamilla 2015; Shree 2011; Nakasone 2012]. TAM survival and function have been shown to be dependent on signaling through MCSF/CSF-1R [DeNardo 2011]. TIE2-expressing macrophages (TEMs) are another subpopulation of tumor-promoting macrophages. TEMs are aggressively pro-angiogenic, pro-metastatic, and immunosuppressive in the tumor microenvironment [Coffelt 2011; Welford 2011; Rigamonti 2014; Ibberson 2013. TAMs and TEMs share many aspects of an M2 pro-tumoral phenotype; however there are some genetic and phenotypic differences. Gene expression profiles have been demonstrated to distinguish TEMs from TAMs, both in the circulation and at tumor sites, highlighting a TEM gene signature that is more proangiogenic [Pucci 2009]. Also, whereas anti-CSF-1R agents are known to ablate TAM populations and block TAM functions, such agents do not ablate a subset of perivascular macrophages that are known to be TIE2Hi [DeNardo 2011; Strachan 2013; Mitchem 2013; Ruffell 2015]. Conversely, anti-ANG/TIE2 agents are known to block perivascular TEM function [De Palma 2005; Mazzieri 2011]. These differences demonstrate a unique susceptibility of TAMs and TEMs to pharmacologic intervention. In addition to their perivascular pro-angiogenic properties, TEMs have been demonstrated to accumulate at the tumor/normal brain interface in glioblastoma tumors and to secrete MMP9 at the tumor invasive front [Gabrusiewicz 2014]. TEMs have also been demonstrated to penetrate into the tumor microenvironment causing dendritic cell anergy and expansion of immunosuppressive Tregs, thus exhibiting an immunomodulation program that favors tumor escape from immune surveillance [Coffelt 2011; Ibberson 2013].

Importantly, a subpopulation of perivascular TEMs compose the Tumor Microenvironment of Metastasis (TMEM) by direct contact with a mammalian enabled (Mena)-expressing tumor cell and an endothelial cell [Robinson 2009; Rohan 2014; Harney 2015]. TMEM are associated with breast cancer metastasis and predict distant recurrence in breast cancer patients independently of other clinical prognostic indicators [Robinson 2009; Rohan 2014]. Mechanistically, a subset of TMEM are composed of TIE2Hi/VEGFAHi TEMs that locally dissolve vascular junctions through VEGFA signaling to mediate local, transient vascular permeability events [Harney 2015]. Motile tumor cells cross the endothelium in association with TIE2Hi/VEGFAHi TMEM macrophages during localized vascular permeability, leading to tumor cell intravasation and resultant circulating tumor cells (CTCs).

The HGF-MET axis has also been shown to play a role in microenvironment-mediated drug resistance. Stromal HGF secretion has been shown to cause resistance to BRAF inhibitor drugs vemurafenib or dabrafenib in melanoma patients [Straussman 2012; Wilson 2012], to cause relapse or refractoriness to anti-VEGF therapy in glioblastoma [Lu 2012; Jahangiri 2013; Piao 2013], and to cause resistance to EGFR inhibitors [Wang 2009; Yano 2011]. MET activation has been demonstrated to induce drug resistance by both tumoral and stromal mechanisms. Treatment of gliomas with anti-VEGF therapies leads to initial tumor responses followed by hypoxia-induced epithelial-to-mesenchymal transition (EMT), a process that renders glioma cells less epithelial and more mesenchymal-like with concomitant increases in invasiveness and resistance. Hypoxia-mediated HIF-1α leads to an up-regulation of MET in these refractory gliomas [Jahangiri 2013]. Anti-VEGF treatment-induced hypoxia also leads to both HGF/MET activation and drug resistance in pancreatic cancer [Kitajima 2008].

Another microenvironment mechanism of drug resistance has been demonstrated wherein HGF/MET activation elicits rebound vascularization during anti-VEGF therapy. MET is expressed on endothelial cells [Ding 2003], and stromal secretion of HGF can lead to MET-mediated angiogenesis in the presence of anti-VEGF therapy [Xin 2001; Van Belle 1998], a phenomenon referred to as evasive revascularization. A dramatic example of VEGF→HGF evasive revascularization has been demonstrated in preclinical models of pancreatic neuroendocrine cancer, wherein initial efficacy of anti-VEGF therapy provokes HGF/MET mediated revascularization and resistance [Sennino 2012; Sennino 2013]. In these preclinical settings, combination therapy comprising VEGF and MET inhibition affords more durable responses and mitigates single agent anti-VEGF mediated revascularization and metastasis.

### SUMMARY OF THE INVENTION

In one aspect, the present disclosure provides methods for treating cancer comprising administering to a subject in need thereof an effective amount of N-(4-(2-(cyclopropanecarboxamido)pyridin-4-yloxy)-2,5-difluorophenyl)-N′-(4-fluorophenyl)cyclopropane-1,1-dicarboxamide, or a pharmaceutically acceptable salt thereof. In some embodiments, the cancer is selected from the group consisting of solid tumors, gastrointestinal stromal tumors, glioblastoma, melanoma, ovarian cancer, breast cancer, renal cancer, hepatic cancer, cervical carcinoma, non small cell lung cancer, mesothelioma, and colon cancer. In some embodiments, the effective amount of N-(4-(2-(cyclopropanecarboxamido)pyridin-4-yloxy)-2,5-difluorophenyl)-N′-(4-fluorophenyl)cyclopropane-1,1-dicarboxamide, or pharmaceutically acceptable salt thereof is administered to the subject orally.

In some embodiments, the present disclosure provides a method for treating cancer by inhibiting tumor cell interactions with the microenvironment comprising administering to a patient in need thereof an effective amount of N-(4-(2-(cyclopropanecarboxamido)pyridin-4-yloxy)-2,5-difluorophenyl)-N′-(4-fluorophenyl)cyclopropane-1,1-dicarboxamide, or a pharmaceutically acceptable salt thereof. The present disclosure also provides a method for treating cancer by inhibiting signaling through the angiopoietin (ANG)-TIE2 kinase signaling axis in the tumor microenvironment, comprising administering to a patient in need thereof an effective amount of N-(4-(2-(cyclopropanecarboxamido)pyridin-4-yloxy)-2,5-difluorophenyl)-N′-(4-fluorophenyl)cyclopropane-1,1-dicarboxamide, or a pharmaceutically acceptable salt thereof.

The present disclosure also provides a method for treating cancer by inhibiting signaling through three microenvironment (re)vascularization and drug resistance pathways (ANG-TIE2, HGF-MET, VEGF-VEGFR2). In some embodiments, the method comprises administering to a patient in need thereof an effective amount of N-(4-(2-(cyclopropanecarboxamido)pyridin-4-yloxy)-2,5-difluorophenyl)-N′-(4-fluorophenyl)cyclopropane-1,1-dicarboxamide, or a pharmaceutically acceptable salt thereof.

The present disclosure also provides a method for treating cancer by inhibiting tumor cell interactions with the microenvironment, comprising administering to a patient in need thereof an effective amount of N-(4-(2-(cyclopropanecarboxamido)pyridin-4-yloxy)-2,5-difluorophenyl)-N′-(4-fluorophenyl)cyclopropane-1,1-dicarboxamide, or a pharmaceutically acceptable salt thereof, in combination with additional chemotherapeutic agents, targeted therapeutic agents, immunotherapy agents, and/or anti-angiogenic therapies.

In some embodiments, the additional chemotherapeutic agent is an anti-tubulin agent. In further embodiments, the anti-tubulin agent is selected from paclitaxel, docetaxel, abraxane, and eribulin.

In some embodiments, the additional anti-cancer targeted therapeutic agent is a kinase inhibitor. For example, in some embodiments, the anti-cancer targeted therapeutic agent is a BRAF kinase inhibitor. In further embodiments, the BRAF inhibitor is dabrafenib or vemurafenib.

In some embodiments, the immunotherapy agent is an anti-CTLA-4 agent, an anti-PD agent, an anti-PDL agent, or an IDO inhibitor. In some embodiments, the immunotherapy agent is selected from ipilimumab, pembrolizumab, nivolumab, atezolizumab, avelumab, MEDI4736, indoximod, INCB024360, and epacadostat.

In some embodiments, the anti-angiogenic agent is bevacizumab.

### BRIEF DESCRIPTION OF THE DRAWINGS

FIG. 1 shows that Compound 1 inhibited TIE2 expressing macrophage (TEM)-mediated tumor cell intravasation in vitro. The left panel of FIG. 1 is a schematic of the assay. The right panel of FIG. 1 shows the instravasation-directed transendothelial migration (iTEM) activity of 10 nM or 100 nM of Compound 1 or control, relative to background.

FIG. 2 shows that Compound 1 inhibited TIE2-mediated capillary tube formation stimulated by 200 ng/mL angiopoeitin-2 (ANG2). The top panel of FIG. 2 shows capillary tube formation in the presence of control DMSO+ANG2. The four panels in the middle of FIG. 2 show capillary tube formation in the presence of, from left to right, (i) 1 nM Compound 1+ANG2, (ii) 10 nM Compound 1+ANG2, (iii) 100 nM Compound 1+ANG2, and (iv) 1000 nM Compound 1+ANG2. As shown in the bottom panel of FIG. 2, the IC50 of Compound 1 in this study was 7 nM.

FIGS. 3A to 3F show that Compound 1 restored inhibition of proliferation in BRAF V600E melanoma cell lines made resistant by stromal HGF. FIG. 3A shows inhibition of proliferation with dabrafenib alone. FIGS. 3B and 3C show treatment with dabrafenib in combination with HGF or with MRC-5 conditioned medium, respectively, which each render dabrafenib ineffective. FIG. 3D shows inhibition of proliferation by Compound 1 alone. FIGS. 3E and 3F show that Compound 1 restored sensitivity of the cells to dabrafenib in the presence of HGF or MRC-5 conditioned medium, respectively.

FIGS. 4A and 4B show that Compound 1 restored the inhibition of signal transduction (pMET and pERK) in SK-MEL-28 (FIG. 4A) or SK-MEL-5 (FIG. 4B) melanoma cells that are resistant to dabrafenib treatment by stromal HGF.

FIG. 5 shows that Compound 1 inhibited orthotopic glioblastoma tumor growth in mice, alone or in combination with bevacizumab.

FIG. 6 shows that Compound 1, alone or in combination with bevacizumab, extends survival of mice in an orthotopic glioblastoma xenograft model. The median survival for placebo control animals was 68 days. The median survival for animals treated with bevacizumab (10 mg/kg IP) was 88 days. The median survival for animals receiving Compound 1 alone (10 mg/kg, twice per day, PO) was 112 days. The median survival for animals receiving Compound 1 (10 mg/kg, twice per day, PO) in combination with bevacizumab was 166 days.

FIG. 7 shows that the combination of Compound 1 with bevacizumab (Bev) decreases circulating TIE2+ and TIE2+/MET+ monocytes. The percent of CD11b+/Gr1-/TIE2+ cells (white bar) and percent of CD11b+/Gr1-/TIE2+MET+ (black bar) are shown.

FIGS. 8A and 8B show that Compound 1 in combination with bevacizumab blocks glioblastoma tumor growth in the GSC11 glioma stem cell xenograft model. FIG. 8A shows the tumor volume at 3 weeks, 4 weeks, and 5 weeks following treatment with bevacizumab, Compound 1, or the combination of bevacizumab with Compound 1. FIG. 8B is a line graph showing the quantified tumor volume in each group (mm³).

FIGS. 9A and 9B show that Compound 1 in combination with bevacizumab blocks glioblastoma tumor growth in the GSC-17 glioma stem cell xenograft model. FIG. 9A shows the tumor volume at 3.5 weeks, 4.5 weeks, and 5.5 weeks following treatment with bevacizumab, Compound 1, or the combination of bevacizumab with Compound 1. FIG. 9B is a line graph showing the quantified tumor volume in each group (mm³).

FIGS. 10A and 10B show that Compound 1 blocked bevacizumab-mediated increase in the presence of the mesenchymal tumor marker vimentin in the GSC11 (FIG. 10A) and GSC17 (FIG. 10B) xenograft mouse models. The left side of both FIG. 10A and FIG. 10B show immunohistochemical staining for vimentin in the tumor following treatment with negative control, bevacizumab, Compound 1, or the combination of bevacizumab with Compound 1. The right side of both FIG. 10A and FIG. 10B is a bar graph showing the quantification of the immunohistochemical staining.

FIGS. 11A and 11B show that Compound 1 blocked bevacizumab-mediated invasiveness and expression of the glioma stem cell marker Nestin in the GSC11 (FIG. 11A) and GSC17 (FIG. 11B) xenograft mouse models. The left sides of both FIG. 11A and FIG. 11B show immunohistochemical staining for Nestin in the tumor following treatment with negative control, bevacizumab, Compound 1, or the combination of bevacizumab with Compound 1. The right side of both FIG. 11A and FIG. 11B is a bar graph showing the quantification of the immunohistochemical staining.

FIGS. 12A and 12B show that Compound 1 reduced the presence of vascular marker Factor VIII in tumors in the GSC11 (FIG. 12A) and GSC17 (FIG. 12B) xenograft mouse models. The left sides of both FIG. 12A and FIG. 12B show immunohistochemical staining for Factor VIII in the tumor following treatment with negative control, bevacizumab, Compound 1, or the combination of bevacizumab with Compound 1. The right side of each of FIG. 12A and FIG. 12B is a bar graph showing the quantification of the immunohistochemical staining.

FIG. 13 shows that Compound 1 reduced the bevacizumab-induced infiltration of F4/80+ monocytes into tumors in the GSC-11 glioma xenograft model. The top set of panels in FIG. 13 show immunohistochemical staining for F4/80. The bottom panel of FIG. 13 is a bar graph showing the quantification of the immunohistochemical staining.

FIG. 14 shows that Compound 1 reduced the bevacizumab-induced infiltration of TIE2+/F4/80+ monocytes into tumors in the GSC-17 glioma xenograft model. The top panels of FIG. 14 show immunohistochemical DAPI staining for nuclei, TIE2 staining, and F4/80 staining for macrophages in the presence of control, bevacizumab, Compound 1, or the combination of bevacizumab and Compound 1. The bottom panel of FIG. 14 is a bar graph showing the quantification of the immunohistochemical staining.

FIG. 15 shows that Compound 1 alone and in combination with paclitaxel reduced mammary breast tumor growth. The mean tumor burden (mg) over time is shown following treatment with vehicle control, 10 mg/kg paclitaxel (every 5 days for 5 administrations, intravenous) Compound 1 (15 mg/kg twice per day, oral), or the combination of paclitaxel and Compound 1.

FIG. 16 shows that Compound 1 along and in combination with paclitaxel resulted in decreased TIE2-expressing macrophage infiltration in the PyMT primary tumor model. The TIE2 score is shown for control, paclitaxel treatment, Compound 1 treatment, or the combination of Compound 1 and paclitaxel.

FIG. 17 shows that Compound 1 blocked lung metastases in the PyMT mammary breast cancer model. Metastases per lung (as a percent of vehicle control) are shown for paclitaxel, Compound 1, or the combination of Compound 1 and paclitaxel.

FIG. 18 shows that Compound 1 exhibited tumor microenvironment efficacy in an A375 melanoma xenograft model and decreases tumor microvessel area. The left panel of FIG. 18 shows the mean tumor burden over time following treatment with vehicle control, 20 mg/kg each day of Compound 1, or 10 mg/kg twice per day of Compound 1. Drugs were dosed on days 8-22 of the study. The right panel of FIG. 18 shows the percent microvessel area following treatment with 20 mg/kg or 10 mg/kg Compound 1.

FIG. 19 shows that Compound 1 (20 mg/kg twice daily, orally or 10 mg/kg twice per day, orally) reduced mean tumor volume in an SKOV3 ovarian xenograft model, relative to vehicle control; the combination of Compound 1 with paclitaxel (10 mg/kg every 5 days for 5 treatments, intravenous) further reduced the mean tumor volume. Drugs were dosed on days 14-42.

### DETAILED DESCRIPTION

In one aspect, compound 1 exhibits balanced inhibition of TIE2, MET, and VEGFR2 kinases within a single therapeutic. This kinase inhibitory profile addresses multiple characteristics of the hallmarks of cancer, including TIE2-, MET-, or VEGFR2-mediated tumor microenvironment mechanisms of angiogenesis, paracrine activation of tumor cells, invasion, metastasis, inflammation, and tumor immunotolerance. Compound 1 is an agent which inhibits three major microenvironment (re)vascularization and drug resistance pathways (ANG, HGF, VEGF), that signal through receptor tyrosine kinases (TIE2, MET, VEGFR2, respectively) and blocks tumor invasion and metastasis.

Because Compound 1 blocks mechanisms of resistance to other treatment modalities, Compound 1 also finds utility in combination with these other treatment modalities. Compound 1 finds utility in combination with anti-VEGF therapies including but not limited to bevacizumab, and in combination with chemotherapies including but not limited to anti-tubulin agents such as paclitaxel, docetaxel, abraxane, or eribulin, or to targeted therapeutic agents including but not limited to the BRAF inhibitor dabrafenib. Compound 1, because of its inhibition of tumor immunotolerant TIE2 expressing macrophages, finds utility in combination with other immunotherapeutic agents including but not limited to an anti-CTLA-4 agent, an anti-PD agent, an anti-PDL agent, or an IDO inhibitor, including but not limited to pembrolizumab, nivolumab, ipilimumab, atezolizumab, avelumab, or MEDI4736, indoximod, INCB024360, or epacadostat.

### Definition

Compound 1 as used herein refers to the compound N-(4-(2-(cyclopropanecarboxamido)pyridin-4-yloxy)-2,5-difluorophenyl)-N′-(4-fluorophenyl)cyclopropane-1,1-dicarboxamide, or a pharmaceutically acceptable salt thereof, whose structure is below:

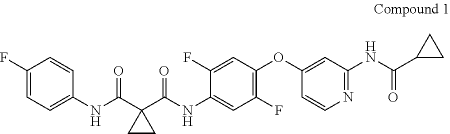 [SMILES: O=C(Nc1cc(Oc2cc(F)c(NC(=O)C3(C(=O)Nc4ccc(F)cc4)CC3)cc2F)ccn1)C1CC1]

Methods of making Compound 1 are disclosed in U.S. Pat. No. 8,637,672 the contents of which are incorporated herein by reference. The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

For convenience, certain terms employed in the specification, examples and claims are collected here. Unless defined otherwise, all technical and scientific terms used in this disclosure have the same meanings as commonly understood by one of ordinary skill in the art to which this disclosure belongs. The initial definition provided for a group or term provided in this disclosure applies to that group or term throughout the present disclosure individually or as part of another group, unless otherwise indicated.

In some embodiments, the present disclosure provides methods for treating cancer comprising administering to a subject in need thereof an effective amount of N-(4-(2-(cyclopropanecarboxamido)pyridin-4-yloxy)-2,5-difluorophenyl)-N′-(4-fluorophenyl)cyclopropane-1,1-dicarboxamide, or a pharmaceutically acceptable salt thereof. In further embodiments, the effective amount of N-(4-(2-(cyclopropanecarboxamido)pyridin-4-yloxy)-2,5-difluorophenyl)-N′-(4-fluorophenyl)cyclopropane-1,1-dicarboxamide, or a pharmaceutically acceptable salt thereof, is administered to the subject in combination with an additional therapeutic agent such as a chemotherapeutic agent, a kinase inhibitor, an immunotherapy agent, and/or an anti-angiogenic agent.

The chemotherapeutic agents may include, but are not limited, to anti-tubulin agents (paclitaxel, paclitaxel protein-bound particles for injectable suspension, eribulin, docetaxel, ixabepilone, vincristine), vinorelbine, DNA-alkylating agents (including cisplatin, carboplatin, oxaliplatin, cyclophosphamide, ifosfamide, temozolomide), DNA intercalating agents (including doxorubicin, pegylated liposomal doxorubicin, daunorubicin, idarubicin, and epirubicin), 5-fluorouracil, capecitabine, cytarabine, decitabine, 5-aza cytadine, gemcitabine and methotrexat.

The kinase inhibitors may include, but are not limited to, erlotinib, gefitinib, lapatanib, everolimus, temsirolimus, LY2835219, LEEO11, PD 0332991, crizotinib, cabozantinib, sunitinib, pazopanib, sorafenib, regorafenib, axitinib, dasatinib, imatinib, nilotinib, vemurafenib, dabrafenib, trarnetinib, idelalisib, and quizartinib.

The immunotherapy agents may include, but are not limited to, anti-CTLA-4 agents, anti-PD agents, anti-PDL agents, IDO inhibitors, ipilimumab, pembrolizumab, nivolumab, atezolizumab, avelumab, MEDI4736, indoximod, INCB024360, and epacadostat.

The anti-angiogenic agents may include, but are not limited to, bevacizurnab, ranibizumab, ramucirumab and aflibercept.

In some embodiments, the cancer is selected from gastrointestinal stromal tumor, glioblastoma, melanoma, ovarian cancer, renal cancer, hepatic cancer, cervical carcinoma, non small cell lung cancer, mesothelioma, colon cancer, colorectal cancer, head and neck cancer, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, pancreatic cancer, breast cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilms' tumor, testicular tumor, lung carcinoma, bladder carcinoma, epithelial carcinoma, glioma, astrocytoma, medulloblastoma, craniopharyngioma, ependymoma, pinealoma, hem angi oblastoma, acoustic neuroma, oligodendroglioma, meningioma, neuroblastoma, retinoblastoma, and neuroendocrine tumor.

The terms “patient” and “subject” are used interchangeably herein. In one embodiment, the subject may be a mammal, such as a rodent, a feline, a canine, and a primate. Preferably, a subject is a human. In one embodiment, the compounds and additional therapeutics provided herein may be administered by any suitable route, independently selected from oral, parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intratympanic, intrauterine, intravesical, intravitreal,bolus, subconjunctival, vaginal, rectal, buccal, sublingual, intranasal, intratumoral, and transdermal. In further embodiments, the effective amount of N-(4-(2-(cyclopropanecarboxamido)pyridin-4-yloxy)-2,5-difluorophenyl)-N′-(4-fluorophenyl)cyclopropane-1,1-dicarboxamide, or a pharmaceutically acceptable salt thereof, is administered to the subject orally.

The term “pharmaceutically acceptable salt” embraces salts commonly used to form salts of free bases. The nature of the salt is not critical, provided that it is pharmaceutically-acceptable. The phrase “pharmaceutically acceptable” is employed in this disclosure to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Suitable pharmaceutically acceptable acid addition salts may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, arylaliphatic, and heterocyclyl containing carboxylic acids and sulfonic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The term “treating” with regard to a subject, refers to improving at least one symptom of the subject's disorder. Treating can be preventing, curing, improving, or at least partially ameliorating the disorder.

The terms “effective amount” and “therapeutically effective amount” are used interchangeably in this disclosure and refer to an amount of a compound that, when administered to a subject, is capable of reducing a symptom of a disorder in a subject. The actual amount which comprises the “effective amount” or “therapeutically effective amount” will vary depending on a number of conditions including, but not limited to, the particular disorder being treated, the severity of the disorder, the size and health of the patient, and the route of administration. A skilled medical practitioner can readily determine the appropriate amount using methods known in the medical arts.

### Biological Data

It has been unexpectedly found that Compound 1 potently inhibits TIE2 kinase biochemically, in whole cell studies, and in in vivo cancer models. The following examples disclose that Compound 1 inhibits TIE2 kinase in two cellular compartments of the tumor microenvironment: 1) vascular endothelial cells; and 2) pro-tumoral macrophages. Additionally it has been found that combinations of Compound 1 with other cancer therapies, including chemotherapeutic agents, targeted therapeutics, and anti-angiogenic agents, overcomes resistance mounted to those other cancer therapies and/or provides superior anti-cancer efficacy.

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### EXAMPLES

### Example 1

### Evaluation of Compound 1 in a Biochemical Assay for uTIE2 Kinase (SEQ ID No. 1)

Activity of uTIE2 kinase was determined by following the production of ADP from the kinase reaction through coupling with the pyruvate kinase/lactate dehydrogenase system [Schindler 2000]. In this assay, the oxidation of NADH (thus the decrease at A_{340 nm}) was continuously monitored spectrophotometrically. The reaction mixture (100 μL) contained TIE2 (SignalChem) (5.6 nM), BSA (0.004% (w/v)), polyEY (1.5 mg/ml), MgCl₂ (15 mM), DTT (0.5 mM), pyruvate kinase (4 units), lactate dehydrogenase (7 units), phosphoenol pyruvate (1 mM), and NADH (0.28 mM) and ATP (1.5 mM) in 90 mM Tris buffer containing 0.2% octyl-glucoside and 1% DMSO, pH 7.5. The inhibition reaction was started by mixing serial diluted test Compound 1 with the above reaction mixture. The absorption at 340 nm was monitored continuously for 6 hours at 30° C. on a plate reader (BioTek). The reaction rate was calculated using the 5 to 6 h time frame. Percent inhibition was obtained by comparison of reaction rate with that of a control (i.e. with no test compound). IC₅₀ value for Compound 1 was calculated from a series of percent inhibition values determined at a range of inhibitor concentrations using software routines as implemented in the GraphPad Prism software package.

Compound 1 inhibited uTIE2 kinase with an IC50 of 7.9 nM.

uTIE2 protein sequence used for screening (SEQ ID No. 1)

QLKRANVQRRMAQAFQNVREEPAVQFNSGTLALNRKVKNNPDPTIYPVLD
WNDIKFQDVIGEGNFGQVLKARIKKDGLRMDAAIKRMKEYASKDDHRDFA
GELEVLCKLGHHPNIINLLGACEHRGYLYLAIEYAPHGNLLDFLRKSRVL
ETDPAFAIANSTASTLSSQQLLHFAADVARGMDYLSQKQFIHRDLAARNI
LVGENYVAKIADFGLSRGQEVYVKKTMGRLPVRWMAIESLNYSVYTTNSD
VWSYGVLLWEIVSLGGTPYCGMTCAELYEKLPQGYRLEKPLNCDDEVYDL
MRQCWREKPYERPSFAQILVSLNRMLEERKTYVNTTLYEKFTYAGIDCSA
EEAA

### Example 2

### Evaluation of Compound 1 in a Biochemical Assay for MET Kinase (SEQ ID No. 2)

Activity of MET kinase (SEQ ID No. 2) was determined by following the production of ADP from the kinase reaction through coupling with the pyruvate kinase/lactate dehydrogenase system [Schindler 2000]. In this assay, the oxidation of NADH (thus the decrease at A340 nm) was continuously monitored spectrophotometrically. The reaction mixture (100 μl) contained MET (MET residues: 1050-1360, from Decode Biostructures, 7 nM), polyE4Y (1 mg/mL), MgCl₂ (17 mM), pyruvate kinase (4 units), lactate dehydrogenase (7 units), phosphoenol pyruvate (1 mM), and NADH (0.28 mM) in 90 mM Tris buffer containing 1 mM DTT, 0.2% octyl-glucoside and 1% DMSO, pH 7.5. Test Compound 1 was incubated with MET (SEQ ID No. 2) and other reaction reagents at 22° C. for 0.5 h before ATP (100 μM) was added to start the reaction. The absorption at 340 nm was monitored continuously for 2 hours at 30° C. on a plate reader (BioTek). The reaction rate was calculated using the 1.0 to 2.0 h time frame. Percent inhibition was obtained by comparison of reaction rate with that of a control (i.e., with no test compound). IC₅₀ values were calculated from a series of percent inhibition values determined at a range of inhibitor concentrations using software routines as implemented in the GraphPad Prism software package.

MET Kinase (SEQ ID No. 2)

TVHIDLSALNPELVQAVQHVVIGPSSLIVHFNEVIGRGHFGCVYHGTLLD
NDGKKIHCAVKSLNRITDIGEVSQFLTEGIIMKDFSHPNVLSLLGICLRS
EGSPLVVLPYMKHGDLRNFIRNETHNPTVKDLIGFGLQVAKGMKYLASKK
FVHRDLAARNCMLDEKFTVKVADFGLARDMYDKEYYSVHNKTGAKLPVKW
MALESLQTQKFTTKSDVWSFGVLLWELMTRGAPPYPDVNTFDITVYLLQG
RRLLQPEYCPDPLYEVMLKCWHPKAEMRPSFSELVSRISAIFSTFIGEHY
VHVNATYVNVK.

Compound 1 inhibited MET kinase with an IC50 of 2.7 nM.

### Example 3

### Evaluation of Compound 1 in a Biochemical Assay for VEGFR2 Kinase (SEQ ID No. 3)

The activity of VEGFR2 kinase was determined by following the production of ADP from the kinase reaction through coupling with the pyruvate kinase/lactate dehydrogenase system [Schindler 2000]. In this assay, the oxidation of NADH (thus the decrease at A340 nm) was continuously monitored spectrophotometrically. The reaction mixture (100 μl) contained VEGFR2 (SEQ ID No. 3, 2.7 nM, nominal concentration), polyE4Y (2.5 mg/mL), pyruvate kinase (4 units), lactate dehydrogenase (7 units), phosphoenolpyruvate (1 mM), and NADH (0.28 mM) in 90 mM Tris buffer containing 0.2% octyl-glucoside, 18 mM MgCl₂, 1 mM DTT, and 1% DMSO at pH 7.5. The reaction was initiated by adding ATP (0.2 mM, final concentration). The absorption at 340 nm was continuously monitored for 3 h at 30° C. on a plate reader (Biotek) or instrument of similar capacity. The reaction rate was calculated using the 2 h to 3 h time frame. Percent inhibition was obtained by comparison of reaction rate with that of a control (i.e., with no test compound). IC₅₀ values were calculated from a series of percent inhibition values determined at a range of Compound 1 concentrations using software routines as implemented in the GraphPad Prism software package.

VEGFR2 Protein Sequence used for Screening (SEQ ID No. 3)

DPDELPLDEHCERLPYDASKWEFPRDRLKLGKPLGRGAFGQVIEADAFGI
DKTATCRTVAVKMLKEGATHSEHRALMSELKILIHIGHHLNVVNLLGACT
KPGGPLMVIVEFCKFGNLSTYLRSKRNEFVPYKVAPEDLYKDFLTLEHLI
CYSFQVAKGMEFLASRKCIHRDLAARNILLSEKNVVKICDFGLARDIYKD
PDYVRKGDARLPLKWMAPETIFDRVYTIQSDVWSFGVLLWEIFSLGASPY
PGVKIDEEFCRRLKEGTRMRAPDYTTPEMYQTMLDCWHGEPSQRPTFSEL
VEHLGNLLQANAQQD

Compound 1 inhibited VEGFR2 kinase with an IC50 of 9.2 nM.

### Example 4

### Evaluation of Compound 1 in TIE2 Expressing CHO-K1 Cell Culture

CHO-K1 cells (catalog #CCL-61) were obtained from the American Type Culture Collection (ATCC, Manassas, Va.). Briefly, cells were grown in F12K medium supplemented with 10% characterized fetal bovine serum (Invitrogen, Carlsbad, Calif.), 100 units/mL penicillin G, 100 μg/ml streptomycin, and 0.29 mg/mL L-glutamine (Invitrogen, Carlsbad, Calif.) at 37 degrees Celsius, 5% CO₂, and 95% humidity. Cells were allowed to expand until reaching 70-95% confluence at which point they were subcultured or harvested for assay use.

CHO K1 cells (1×10⁵ cells/well) were added to a 24-well tissue-culture treated plate in 1 mL of RPMI1640 medium supplemented with 10% characterized fetal bovine serum and 1× non-essential amino acids (Invitrogen, Carlsbad, Calif.). Cells were then incubated overnight at 37 degrees Celsius, 5% CO₂, and 95% humidity. Medium was aspirated, and 0.5 mL of medium was added to each well. Transfection-grade plasmid DNA (TIE2 gene Gateway cloned into pcDNA3.2™/V5-DEST expression vector, Invitrogen, Carlsbad, Calif.) was diluted to 5 μg/mL in room temperature Opti-MEM® I Medium without serum (Invitrogen, Carlsbad, Calif.). Two μL of Lipofectamine LTX Reagent (Invitrogen, Carlsbad, Calif.) was added per 0.5 μg of plasmid DNA. The tube was mixed gently and incubated for 25 minutes at room temperature to allow for DNA-Lipofectamine LTX complex formation. 100 μL of the DNA-Lipofectamine LTX complex was added directly to each well containing cells and mixed gently. Approximately 18-24 hours post-transfection, medium containing DNA-Lipofectamine complexes was aspirated, cells were washed with PBS, and RPMI1640 medium supplemented with 10% characterized fetal bovine serum (Invitrogen, Carlsbad, Calif.), and 1× non-essential amino acids (Invitrogen, Carlsbad, Calif.) was added. Compound 1 or DMSO was added to the wells (0.5% final DMSO concentration). The plates were then incubated for 4 hours at 37 degrees Celsius, 5% CO₂, and 95% humidity. Following the incubation, the media was aspirated and the cells were washed with PBS. The cells were lysed using MPER lysis buffer (Pierce, Rockford, Ill.) containing Halt Phosphatase and Protease Inhibitors (Pierce, Rockford, Ill.) and Phosphatase inhibitor cocktail 2 (Sigma, St. Louis, Mo.) at 4° C. for 10 minutes with shaking. Cleared lysates were separated by SDS-PAGE on a 4-12% Novex NuPage Bis-Tris gel (Invitrogen, Carlsbad, Calif.) and then transferred to PVDF (Invitrogen, Carlsbad, Calif.). After transfer, the PVDF membrane was blocked with BSA (Santa Cruz Biotechnology, Santa Cruz, Calif.) and then probed with an antibody for phospho-TIE2 (Cell Signaling Technology, Beverly, Mass.). A secondary anti-rabbit antibody conjugated to horseradish peroxidase (Cell Signaling Technology, Beverly, Mass.) was used to detect phospho-TIE2. ECL Plus (GE Healthcare, Piscataway, N.J.), a substrate for horseradish peroxidase that generates a fluorescent product, was added. Fluorescence was detected using a Storm 840 phosphorimager (GE Healthcare, Piscataway, N.J.) in fluorescence mode. PVDF membranes were stripped and then re-probed with total TIE2 antibody (Santa Cruz Biotechnology, Inc., Dallas, Tex.) as above. The 160 kDa phospho-TIE2 and total TIE2 bands were quantified using ImageQuant software (GE Healthcare, Piscataway, N.J.), and data was plotted using Prism software (GraphPad Software, San Diego, Calif.).

Compound 1 exhibited an IC50 value of 2.4 nM for inhibiting the constitutive phosphorylation of TIE2 in Chinese Hamster Ovary (CHO) cells transfected to transiently express high levels of TIE2.

### Example 5

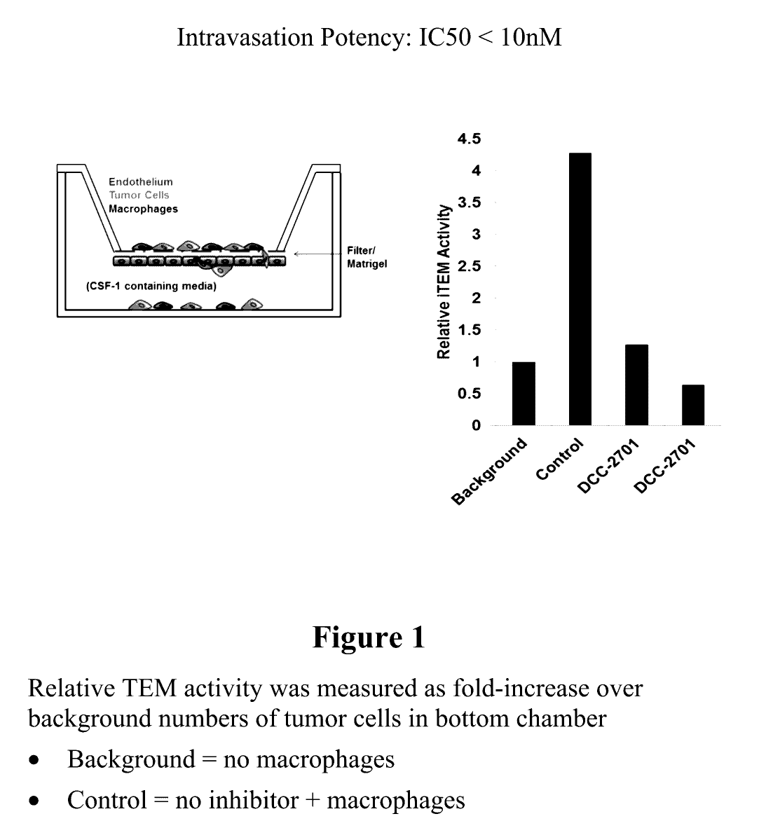

### Evaluation of Compound 1 for Inhibition of TIE2-Expressing Macrophage (TEM)-Mediated Tumor Cell Intravasation (FIG. 1)

In vitro intravasation-directed transendothelial migration (iTEM) assay protocol. The iTEM assay was performed in a Transwell two-chamber apparatus. The Transwell was prepared so that tumor cell transendothelial migration of tumor cells was in the intravasation direction (from subluminal side to luminal side of the endothelium). We define this as the iTEM assay. To prepare the endothelial monolayer, the underside of each Transwell was coated with 50 μl of Matrigel (2.5 μg/ml; Invitrogen). About 100,000 HUVEC cells were plated on the Matrigel-coated underside of the Transwells. Transwells were then flipped onto a 24-well plate containing 200 μl of α-MEM (minimum essential medium) supplemented with 10% fetal bovine serum (FBS)+3000 U of CSF-1 and incubated until the endothelium formed impermeable monolayers. Permeability of both monolayers was tested as described previously by diffusion of 70 kD of Texas Red dextran (Molecular Devices SpectraMax M5 plate reader) and by electrical resistance (World Precision Instruments), which demonstrated that the monolayer was impermeable at 48 hours after plating of the HUVECs; therefore, Transwells were used at this time point. Once impermeable by these criteria, the Transwell assay was used for iTEM studies. All the assays were run in the presence of BAC1.2F5 murine macrophage cell line and MDA-MB 231-GFP human breast cancer cells. Transwells were imaged using a Leica SP5 confocal microscope using a 60×1.4 numerical aperture objective and processed using ImageJ [National Institutes of Health (NIH)] and IMAMS programs. Quantitation was performed by counting the number of tumor cells that had crossed the endothelium within the same field of view (60×, 10 random fields).

Compound 1 inhibited macrophage-mediated intravasation of human breast cancer cells with an IC50 value<10 nM (FIG. 1).

### Example 6

### Evaluation of Compound 1 for Cellular Inhibition of TIE2, MET, and VEGFR2 Kinases in Human Umbilical Vein Endothelial Cells (HUVECs)

### HUVEC Cell Culture

HUVEC (Human umbilical vein endothelial cells; Catalog #CRL-1730) cells were obtained from the American Type Culture Collection (ATCC, Manassas, Va.) for the HUVEC Phospho-TIE2 Western Blot Assay. HUVEC (Catalog #C2519A) cells were obtained from Lonza (Walkersville, Md.) for the HUVEC Enzyme-linked immunosorbent assays. Briefly, cells were grown in EGM-2 media (Lonza, Walkersville, Md.) at 37 degrees Celsius, 5% CO₂, and 95% humidity. Cells were allowed to expand until reaching 90-95% saturation at which point they were subcultured or harvested for assay use.

### HUVEC Phospho-TIE2 Western Blot Assay

HUVEC cells (2.5×10⁵ cells/well) were added to a 24-well tissue-culture treated plate in 1 mL of EGM-2 culture medium (Lonza, Walkersville, Md.). Cells were then incubated overnight at 37 degrees Celsius, 5% CO₂, and 95% humidity. Media was then aspirated and 1 mL EBM-2 basal medium (Lonza, Walkersville, Md.) supplemented with 2% FBS (Invitrogen, Carlsbad, Calif.) was added. Compound 1 or DMSO was added to the wells (0.5% final DMSO concentration). The plates were then incubated for 4 hours at 37 degrees Celsius, 5% CO₂, and 95% humidity. During the incubation, histidine-tagged angiopoietin 1 (ANG1) growth factor (R&D Systems, Minneapolis, Minn.) was added to an anti-polyhistidine antibody (R&D Systems, Minneapolis, Minn.) for 30 minutes at room temperature to generate multimers of ANG1. Following the four hour incubation of Compound 1, cells were stimulated with 800 ng/mL of the ANG1/anti-polyhistidine antibody complex mixture for 15 minutes. The media was aspirated and the cells were washed with PBS. The cells were lysed using MPER lysis buffer (Pierce, Rockford, Ill.) containing Halt Phosphatase and Protease Inhibitors (Pierce, Rockford, Ill.) and Phosphatase inhibitor cocktail 2 (Sigma, St. Louis, Mo.) at 4° C. for 10 minutes with shaking. Cleared lysates were separated by SDS-PAGE on a 4-12% Novex NuPage Bis-Tris gel (Invitrogen, Carlsbad, Calif.) and then transferred to PVDF (Invitrogen, Carlsbad, Calif.). After transfer, the PVDF membrane was blocked with BSA (Santa Cruz Biotechnology, Santa Cruz, Calif.) and then probed with an antibody for phospho-TIE2 (Cell Signaling Technology, Beverly, Mass.). A secondary anti-rabbit antibody conjugated to horseradish peroxidase (Cell Signaling Technology, Beverly, Mass.) was used to detect phospho-TIE2. ECL Plus (GE Healthcare, Piscataway, N.J.), a substrate for horseradish peroxidase that generates a fluorescent product, was added. Fluorescence was detected using a Storm 840 phosphorimager (GE Healthcare, Piscataway, N.J.) in fluorescence mode. The 160 kDa phospho-TIE2 band was quantified using ImageQuant software (GE Healthcare, Piscataway, N.J.). Data was analyzed using Prism software (GraphPad Software, San Diego, Calif.) to calculate the IC₅₀ value.

Compound 1 inhibited ANG1-stimulated phospho-TIE2 in HUVECs with an IC50 of 1.0 nM.

### HUVEC Enzyme-Linked Immunosorbent Assays

HUVECs (250,000 cells/well) were added to 12-well plates in EBM-2 media (Lonza, Inc., Basel, Switzerland) containing 2% FBS. Cells were then incubated overnight. For HUVECs, cells were incubated 4 hr with compound, then stimulated with 200 ng/mL HGF for 15 min. Phospho-MET in cell lysates was detected using an ELISA (R&D Systems). Phospho-VEGFR2 ELISAs were performed as above, except HUVECs were plated in 96-well plates (25,000 cells/well). Cells were incubated overnight, and Compound 1 was then added for 4 hr. Cells were stimulated with 100 ng/mL VEGF (R&D Systems) for 5 min. Phospho-VEGFR2 in cell lysates was detected using an ELISA (R&D Systems).

Compound 1 inhibited HGF-stimulated phospho-MET in HUVECs with an IC50 of IC₅₀ of 2.3 nM.

Compound 1 inhibited VEGF-stimulated phospho-VEGFR2 in HUVECs with an IC50 of 4.7 nM.

### Example 7

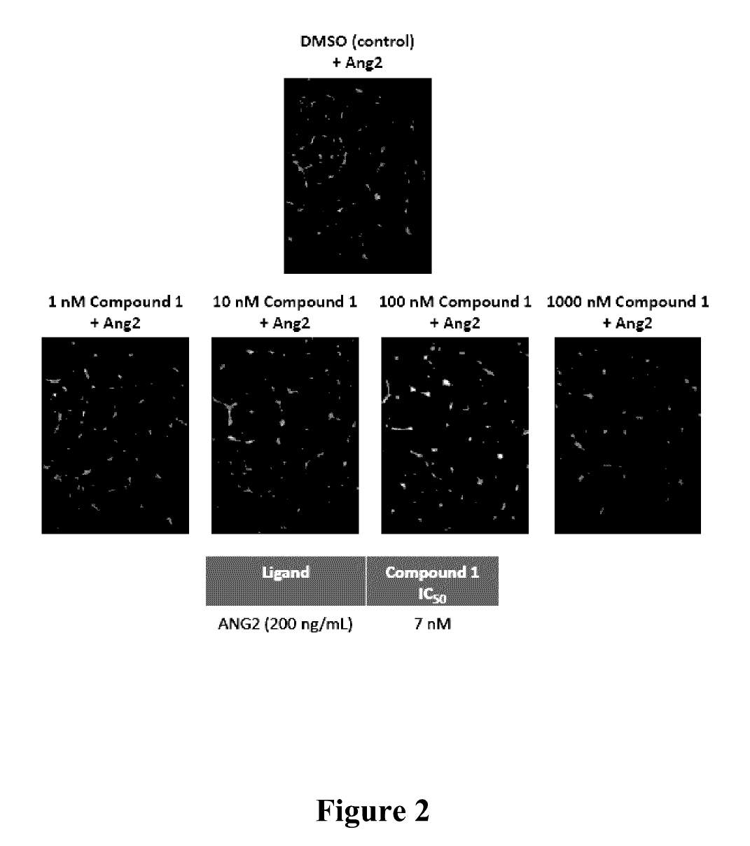

### Evaluation of Compound 1 for Inhibition of Capillary Tube Formation (FIG. 2)

### HMVEC Cell Culture

HMVEC (Human microvascular endothelial cells; Catalog #PCS-110-010) cells were obtained from the American Type Culture Collection (ATCC, Manassas, Va.). Briefly, cells were grown in EGM-2 MV media (Lonza, Walkersville, Md.) at 37 degrees Celsius, 5% CO₂, and 95% humidity. Cells were allowed to expand until reaching 90-95% saturation at which point they were subcultured or harvested for assay use.

Growth factor reduced Matrigel gel solution (BD Biosciences, San Jose, Calif.) was dispensed using pre-chilled tips into each well of a pre-chilled 96-well black, clear bottom, tissue-culture treated plate. The plate was incubated at 37° C. for 1 h to allow the matrix to form a gel. In another 96-well plate, Compound 1 was spotted into each well, followed by the addition of HMVECs (15,000 cells/well in serum-free EBM-2 media; in the presence or absence of 200 ng/mL ANG2, 40 ng/mL HGF, or 100 ng/mL VEGF) to each well. The cell/Compound 1 suspensions were then transferred to the Matrigel plate wells and incubated overnight. The final concentration of DMSO in the assay was 0.5%. The next day, cells were stained with Calcein-AM dye (Life Technologies). Images obtained via fluorescent microscopy were analyzed for tube length using ImagePro Analyzer (Media Cybernetics, Rockville, Md.) with a macro to automatically detect and measure tube formation.

Compound 1 inhibited capillary tube formation with IC₅₀ values of 7 nM, 11 nM, and 58 nM upon stimulation with ANG2, HGF, and VEGF, respectively. FIG. 2 illustrates inhibition of ANG2/TIE2-mediated capillary tube formation.

### Example 8

### Compound 1 Reverses BRAF Therapy Resistance to Dabrafenib Mediated by Stromal HGF

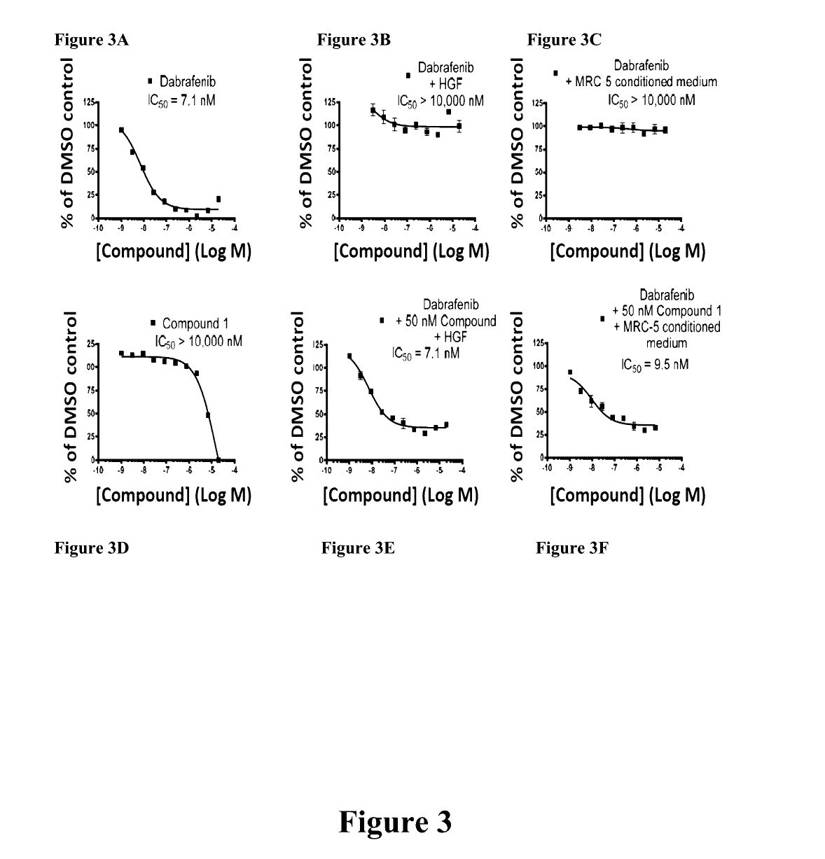

### HGF-Stimulated Melanoma Proliferation Cell Assays (FIG. 3A-3F)

Compound 1 in DMSO was dispensed into assay plates. SK-MEL-28 cells were added to 96-well plates (2,500 cells/well). Effects on cell proliferation were assessed by incubation of SK-MEL-28 cells in complete media, media containing 50 ng/mL HGF, or media mixed 1:1 with MRC-5 fibroblast-conditioned media. Plates were incubated for 72 h. Viable cells were identified by incubation with the vital dye resazurin and quantitated on a plate reader with excitation at 540 nM and emission at 600 nM.

Dabrafenib, a clinically used BRAF kinase inhibitor, potently inhibited proliferation of the SK-MEL 28 mutant BRAF cell line, with an IC₅₀ value of 10.6 nM (representative data shown in FIG. 3A). When HGF or fibroblast-conditioned media containing HGF was added, dabrafenib was rendered ineffective (IC₅₀>10 μM) (FIGS. 3B and 3C). Compound 1 as a single-agent weakly inhibited proliferation of SK-MEL-28 cells, as expected (IC₅₀>10 μM; FIG. 3D). However, the combination of a titration of dabrafenib in the presence of Compound 1 (50 nM) restored sensitivity of the cells to dabrafenib in the presence of HGF or fibroblast-conditioned media (FIGS. 3E and 3F).

Compound 1 restores anti-proliferation sensitivity to dabrafenib in SK-MEL 28 melanoma cells made resistant by stromal HGF (FIG. 3A-F).

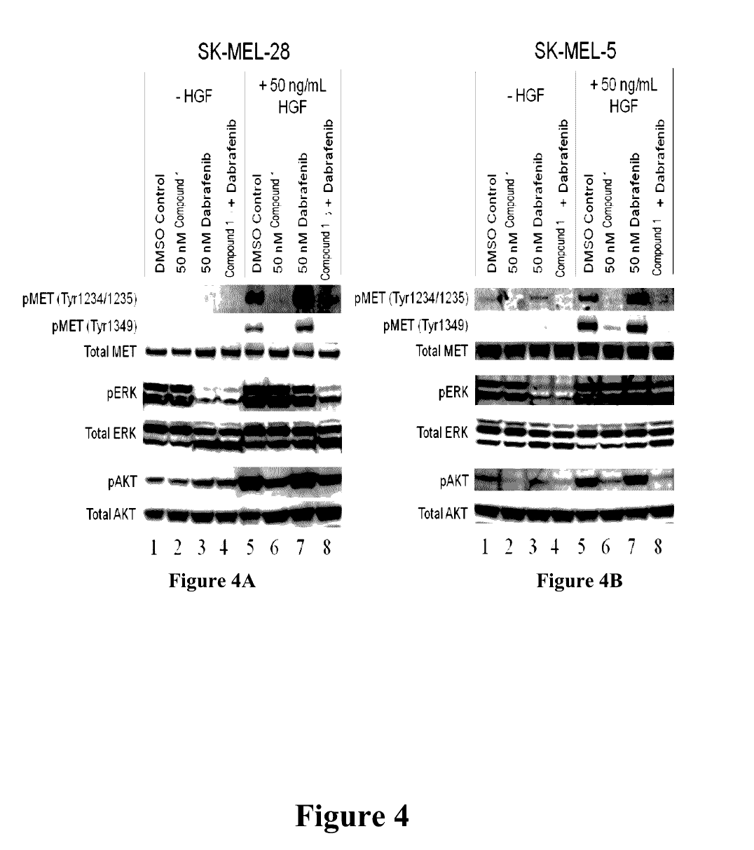

### HGF-Stimulated Melanoma Cell Signal Transduction Assays (FIGS. 4A and 4B)

SK-MEL-5 and SK-MEL-28 cells were added to 12-well plates at 450,000 and 250,000 cells/well, respectively, and incubated overnight. Compound 1 was added and plates incubated for 4 hr. Cells were then stimulated with 50 ng/mL HGF for 1 hr. Antibodies were obtained from Cell Signaling Technology.

Dabrafenib (50 nM) inhibited phosphorylation of the downstream RAF effector ERK in the BRAFV600E melanoma cell lines SK-MEL-28 and SK-MEL-5 (FIGS. 4A and 4B, Lane 3). However, upon HGF stimulation, mimicking a stromal tumor microenvironment resistance mechanism, ERK remained fully phosphorylated in the presence of dabrafenib (FIGS. 4A and 4B, Lane 7). Compound 1 (50 nM) inhibited HGF-induced activation of MET and AKT (FIGS. 4A and 4B, Lane 6), and the addition of Compound 1 to dabrafenib in these models restored complete inhibition of ERK phosphorylation (FIGS. 4A and 4B, Lane 8).

Compound 1 restores inhibition of signal transduction in SK-MEL 28 melanoma cells made dabrafenib resistant by stromal HGF.

### Example 9

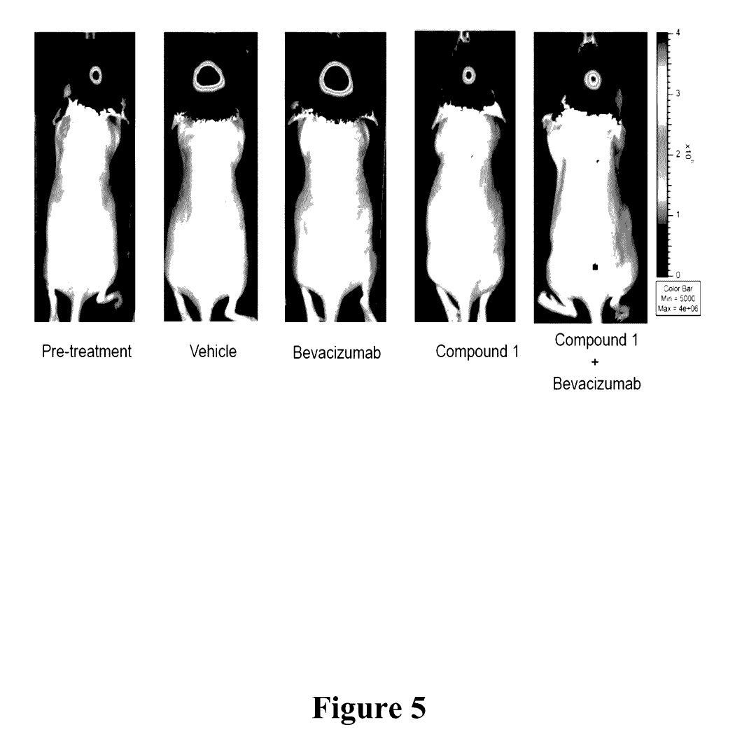

### Evaluation of Compound 1 as a Single Agent and in Combination with Bevacizumab in the Orthotopic U87-MG Xenograft Efficacy Model (FIG. 5)

Protocol for in vivo xenograft efficacy study. Female nude mice were implanted intracranially with one million luciferase-enabled U87-MG cells. Treatments began on Day 31. Brain BLI signal was determined on Day 45 using an IVIS 50 optical imaging system (Xenogen, Alameda, Calif.). Animals were injected IP with 150 mg/kg D-Luciferin and imaged 10 min post-injection.

Compound 1 was evaluated as a single agent and in combination with bevacizumab in the U87-MG xenograft glioma model. The tumor cells were injected intracerebroventricularly (ICV) and tumor growth was monitored in vivo by quantitation of luciferase-mediated BLI.

Bevacizumab treatment (5 mg/kg IP every three days) resulted in a statistically insignificant 63% decrease in BLI signal compared to vehicle after two weeks of treatment. Compound 1 dosed at 10 mg/kg BID led to a significant 90% decrease in BLI signal (p=0.0013). Furthermore, the combination of Compound 1 with bevacizumab resulted in a significant >90% decrease in the BLI signal (p=0.0005; FIG. 5).

### Example 10

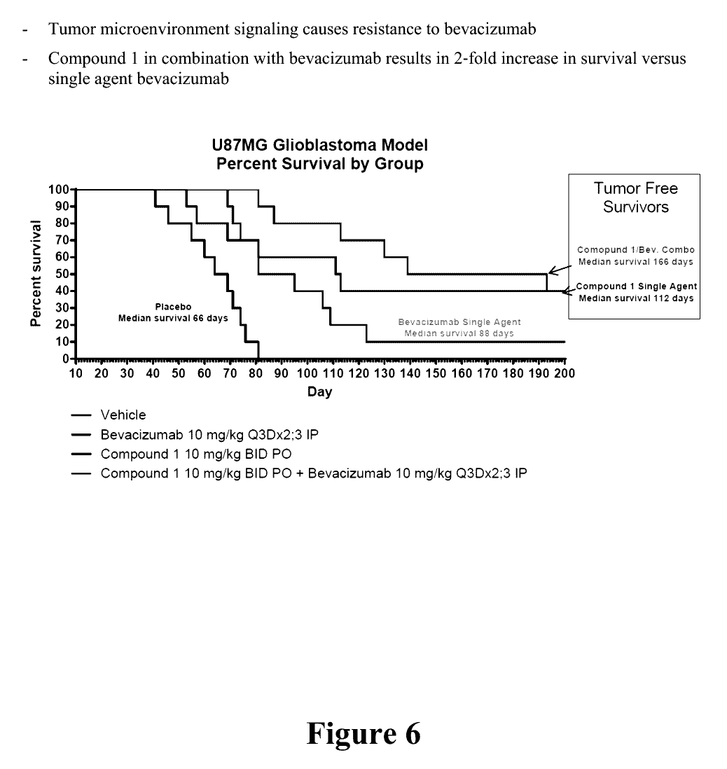

### Evaluation of Compound 1 in the Orthotopic U87-MG Xenograft Survival Model as a Single Agent and in Combination with the Anti-VEGF Therapy Bevacizumab (FIG. 6)

Protocol for in vivo xenograft survival study. Female nude mice were implanted intracranially with one million luciferase-enabled U87-MG cells. Treatments began on Day 12. Mice (n=10/group) were dosed until the end of study. Three additional mice were treated as above for pharmacodynamic analysis after five weeks. Brain BLI signal was determined using an IVIS 50 optical imaging system (Xenogen, Alameda, Calif.). Animals were injected IP with 150 mg/kg D-Luciferin and imaged 10 min post-injection.

Flow cytometry of circulating TIE2/MET-expressing monocytes from U87-MG xenograft model. Peripheral blood samples were fixed using Lyse/Fix buffer (BD Biosciences, San Jose, Calif.). BD Fc Block was added and cells were stained with CD11b-PECy7 and Gr1-APC antibodies (BD Pharmingen), and TIE2-PE and MET-FITC antibodies (eBioscience) or isotype controls. Data was collected on an Accuri C6 cytometer. Monocytes were gated using side and forward scatter. CD11b+/Gr-1-cells were gated, and TIE2- and MET-positive cells were quantified.Compound 1 provides a survival benefit in the U87-MG glioma xenograft model when compared with vehicle control. Compound 1 in combination with bevacizumab provides a survival benefit when compared to single agent bevacizumab.

In this survival study, bevacizumab was dosed as a single agent at 10 mg/kg intraperitoneally twice weekly, Compound 1 was dosed twice daily as a single agent orally at 10 mg/kg, and Compound lwas also administered in combination with bevacizumab on the same dosing schedule as each single agent. Treatment began on Day 12, when the mean brain BLI signal was 5.8×10⁶ photons/sec, and continued until the end of study. Mice were followed through survival. The median survival of vehicle-treated mice was 66.5 days; median survival of the bevacizumab cohort was 88 days (p=0.0013). By comparison, the median survival benefit of Compound 1-treated mice was 112 days (p=0.0047), and in combination with bevacizumab median survival increased further to 166 days (p<0.0001 vs vehicle; p=0.016 vs bevacizumab single agent) (FIG. 6). Thus Compound 1 in combination with bevacizumab resulted in a significant 2.5-fold increase in survival compared to vehicle and a significant 1.9-fold increase in survival compared to bevacizumab alone. In the combination treatment group, ex vivo BLI of brain hemispheres showed three of four survivors were tumor free at the end of study.

Compound 1 alone and in combination with bevacizumab leads to significant increases in overall survival compared to single agent bevacizumab in the U87 ICV glioma model (FIG. 6).

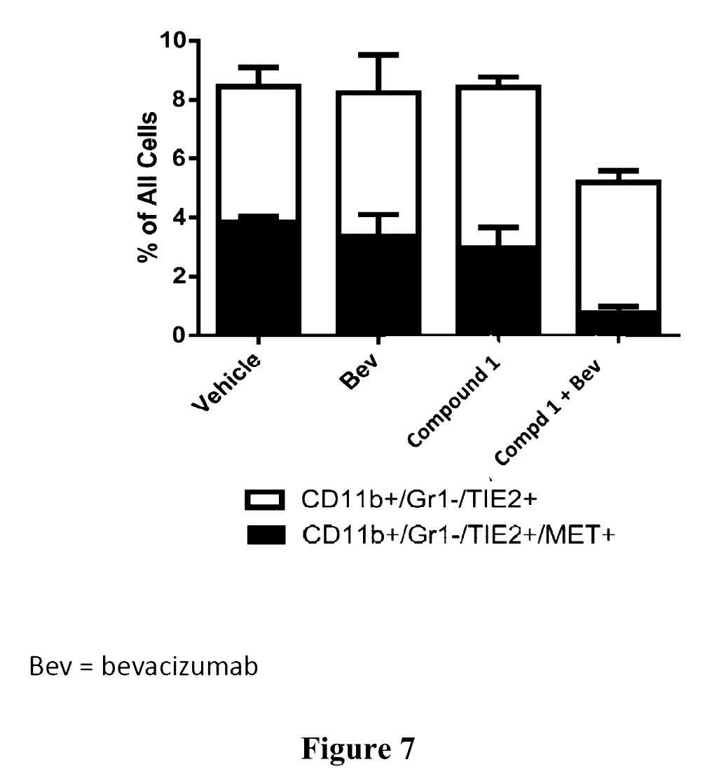

It has been demonstrated previously that certain cancers, including melanomas and gliomas, lead to an increased circulation of TIE2+ circulating monocytes and also to the circulation of “tumor educated” TIE2⁺/MET⁺ pro-angiogenic monocytes [Peinado 2012]. To monitor these monocytes in the U87 survival model, peripheral monocytes were gated on CD11b+/Grl-surface markers and TIE2⁺ and/or TIE2⁺/MET⁺ cells quantitated. In the U87 glioma survival study, we observed both of these TIE2⁺ expressing monocyte populations in the circulation of vehicle-treated mice after five weeks of treatment. Compound 1 and bevacizumab as single agents did not alter the numbers of either circulating monocyte populations. However, combination treatment with Compound 1 and bevacizumab resulted in a 40% decrease in circulating TIE2+ monocytes (p=0.08) and an 80% decrease in proangiogenic TIE2⁺/MET⁺ monocytes (p=0.005, FIG. 7). The lowered population of both of these TIE2⁺ monocyte populations in the combination cohort was consistent with greater survival benefit (FIG. 6).

Compound 1 in combination with bevacizumab decreased circulating levels of TIE2⁺ expressing monocytes (FIG. 7).

### Example 11

### Evaluation of Compound 1 as a Single Agent and in Combination with Bevacizumab in Patient-Derived Xenograft Models of Invasive Glioblastoma

### Patient-Derived Xenograft Study Protocol

For the in vivo experiments, 4- to 6-week-old female nude mice that were strictly inbred at The University of Texas MD Anderson Cancer Center and maintained in the MD Anderson Veterinary in accordance with institutional guidelines were used. For the in vivo experiments, GSC11 (5×10⁵) or GSC17 (5×10⁴) glioblastoma cells were implanted intracranially into nude mice. Beginning 4 days after the implantation, bevacizumab (10 mg/kg) was administered by intraperitoneal (i.p.) injection twice per week in the bevacizumab-only group and in the bevacizumab plus Compound 1 combination group. Compound 1 (10 mg/kg) was administered by oral gavage twice daily in the bevacizumab plus Compound 1 group and in the Compound 1-only group. Control mice for these cohorts were treated with phosphate-buffered saline by i.p. injection and/or with 0.4% HPMC vehicle by oral gavage. One cohort of 10 mice per group were treated continuously and followed for survival. A separate cohort of 9 mice per group were treated continuously until the designated time point, and the tumors from these mice were extracted at 3, 4, 5 weeks in GSC11 xenograft mice model and at 3.5, 4.5, 5.5 weeks in GSC17 xenograft mice model after the start of treatment.

When the mice treated for the designated time period developed signs and symptoms of advanced tumors, they were euthanized, and their brains were removed, fixed in 4% formaldehyde for 24 hours, and embedded in paraffin. The tissues were then sectioned serially (4 μm) and stained with hematoxylin and eosin (Sigma-Aldrich). Tumor formation and phenotype were determined by histologic analysis of the hematoxylin- and eosin-stained sections. The tumor volume, greatest longitudinal diameter (length), and greatest transverse diameter (width) were measured using an external caliper and Adobe Illustrator software. The tumor volumes were calculated by the following formula: volume=½(length×width²).

### Immunohistochemistry and Immunofluorescence

For immunohistochemical analyses, the tissue sections were deparaffinized and subjected to graded rehydration. After blocking the tissue in 5% horse and goat serum and antigen retrieval solution (citrate buffer, pH 6.0), we incubated the tissue sections overnight at 4° C. with primary antibodies against Factor VIII (1:500; A0082, Dako) to assess microvessel formation, vimentin (1:100; M0725, Dako) to assess mesenchymal marker expression, F4/80 (1:50; 123102, BioLegend) to assess monocyte infiltration, nestin (1:300; Ab6142, Abcam) to assess glioma stem cell marker expression, HGF (1:200; LS-B4657, LSBio), and TIE2 (1:50; Santa Cruz Biotechnology) to assess TIE2-expressing monocyte infiltration. Texas red fluorescein isothiocyanate-conjugated secondary antibodies or anti-rat immunoglobulin G antibodies (Invitrogen) were used for 1 hour at room temperature.

### Statistical Analyses

All statistical analyses were conducted with the GraphPad 6 (InStat) software for Windows 7. Survival analysis was conducted using the Kaplan-Meier method, and differences in survival between treatment groups were assessed using the log-rank test. All other comparisons were performed using an unpaired two-tailed Student t-test. Summary statistics for continuous data are expressed as the mean±standard error of the mean. P values less than 0.05 were considered statistically significant. The nestin staining was assessed using the Image-Pro Plus system version 7.0 (Media Cybernetics) in ×10 fields of at least three tumor samples per group with three to four different sections per tumor sample.

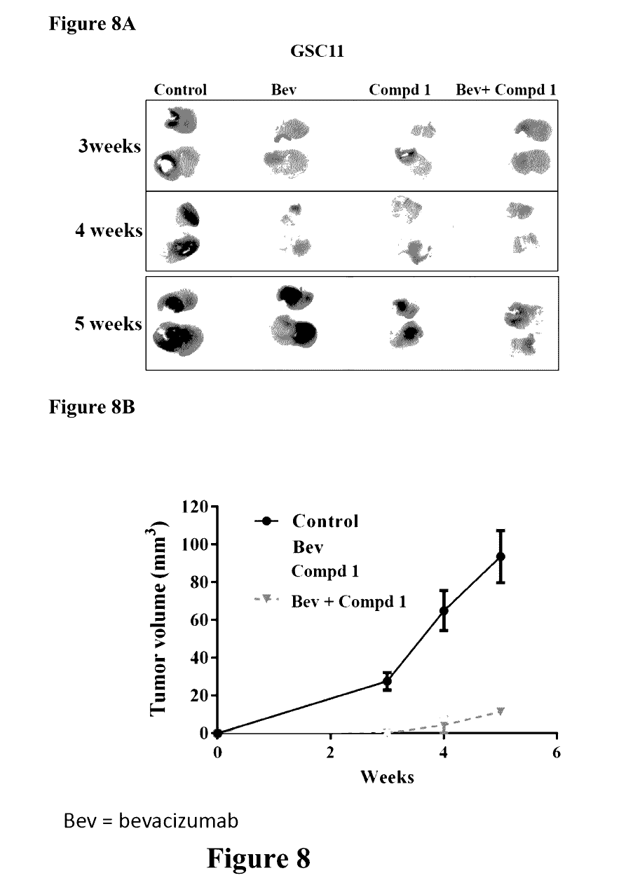

### Compound 1 Blocks Bevacizumab-Mediated Epithelial to Mesenchymal Transition (EMT) and Glioma Tumor Progression (FIGS. 8-11)

FIGS. 8A and 8B demonstrate that Compound 1 in combination with bevacizumab is superior to bevacizumab alone in blocking glioblastoma tumor growth in the human-derived GSC-11 glioma stem cell xenograft model. In the GSC11 model, tumor volumes at 3, 4, and 5 weeks were 27.6±4.5 mm³, 64.9±10.6 mm³, and 93.6±13.7 mm³, respectively, in the control-treated mice and 14.3±6.9 mm³, 30.7±4.2 mm³, and 46.6±4.5 mm³, respectively, in the bevacizumab-treated mice (vs control: p=0.049, p=0.006, and p=0.004889, respectively). Coumpound 1 treatment alone significantly inhibited the tumor volume, which was 1.2±0.5 mm³, 9.8±2.8 mm³, and 32.3±14.6 mm³ at 3, 4, and 5 weeks, respectively (vs control: p=0.000055, p=0.000951, and p=0.00614, respectively). Compound 1 combined with bevacizumab dramatically reduced the tumor volume to 0.1±0.1 mm³, 4.5±4.3 mm³, and 11.4±2.0 mm³ at 3, 4, and 5 weeks, respectively (vs control: p=0.000461, p=0.000788, and p=0.000513, respectively; vs bevacizumab alone: p=0.027, p=0.00169, and p=0.000242, respectively.

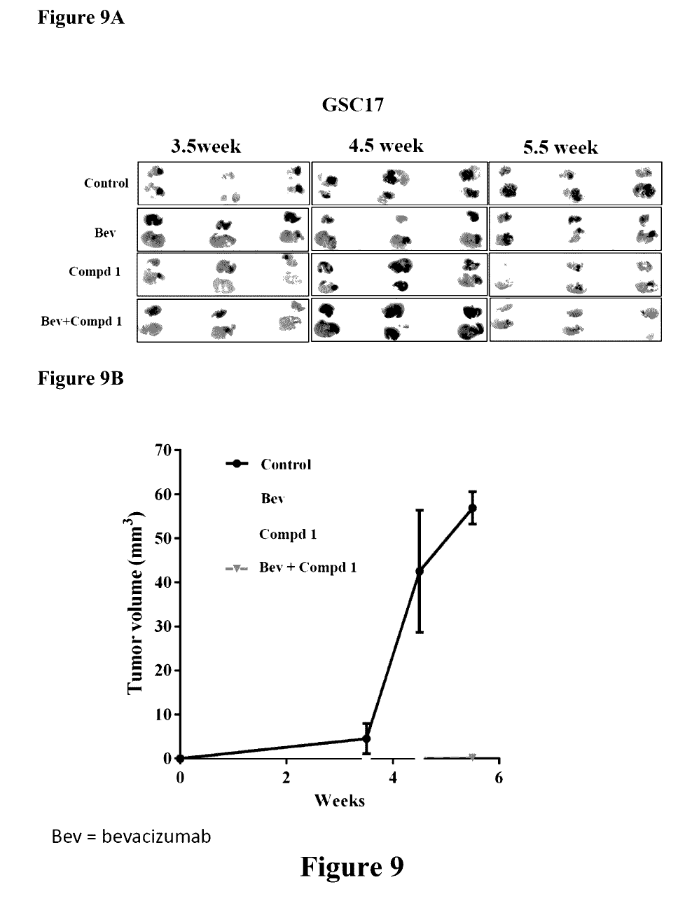

FIGS. 9A and 9B demonstrate that Compound 1 in combination with bevacizumab is superior to bevacizumab alone in blocking glioblastoma tumor growth in the human-derived GSC-17 glioma stem cell xenograft model. At 3.5, 4.5, and 5.5 weeks, the tumor volumes were 4.5±3.4 mm³, 42.5±13.9 mm³, and 56.9±7.4 mm³, respectively, in the control-treated mice and 0.4±0.4 mm³, 0.05±0.03 mm³, and 9.6±4.6 mm³ in the bevacizumab-treated mice (vs control: p=0.105, p=0.0065, and p=0.0001, respectively). Compound 1 treatment alone markedly suppressed tumor volume at 0.1±0.2 mm³, 9.8±3.9 mm³, and 10.1±1.4 mm³ at 3.5, 4.5, and 5.5 weeks, respectively (vs control: p=0.087, p=0.017, and p=0.00003, respectively). Compound 1 combined with bevacizumab dramatically reduced the tumor volume to 0.081±0.1 mm³, 0.04±0.02 mm³, and 0.19±0.11 mm³ at 3.5, 4.5, and 5.5 weeks, respectively (vs control: p=0.085, p=0.0064, and p=0.000013, respectively; vs bevacizumab alone: p=0.28, p=0.58 and p=0.02, respectively.

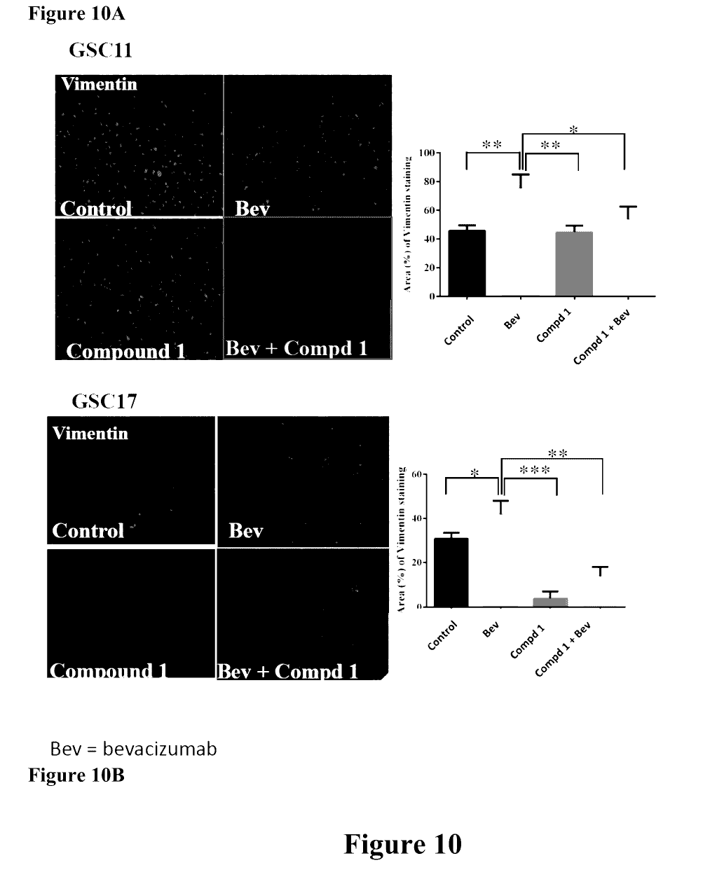

FIG. 10A demonstrates that treatment with the anti-VEGF therapy bevacizumab alone leads to therapy-induced increase in EMT (increase in the mesenchymal tumor marker vimentin) and tumor invasiveness in the GSC-11 glioma model, whereas Compound 1 treatment as a single agent does not cause elevation of vimentin and associated EMT. Moreover, Compound 1 in combination with bevacizumab prevents bevacizumab-induced increases in vimentin and associated EMT.

FIG. 10B demonstrates that treatment with the anti-VEGF therapy bevacizumab alone leads to therapy-induced increase in EMT (increase in the mesenchymal tumor marker vimentin) and tumor invasiveness in the GSC-17 glioma model, whereas Compound 1 treatment as a single agent actually decreases levels of vimentin and associated EMT compared to control and compared to bevacizumab. Moreover, Compound 1 in combination with bevacizumab reverses bevacizumab-induced increases in vimentin and associated EMT.

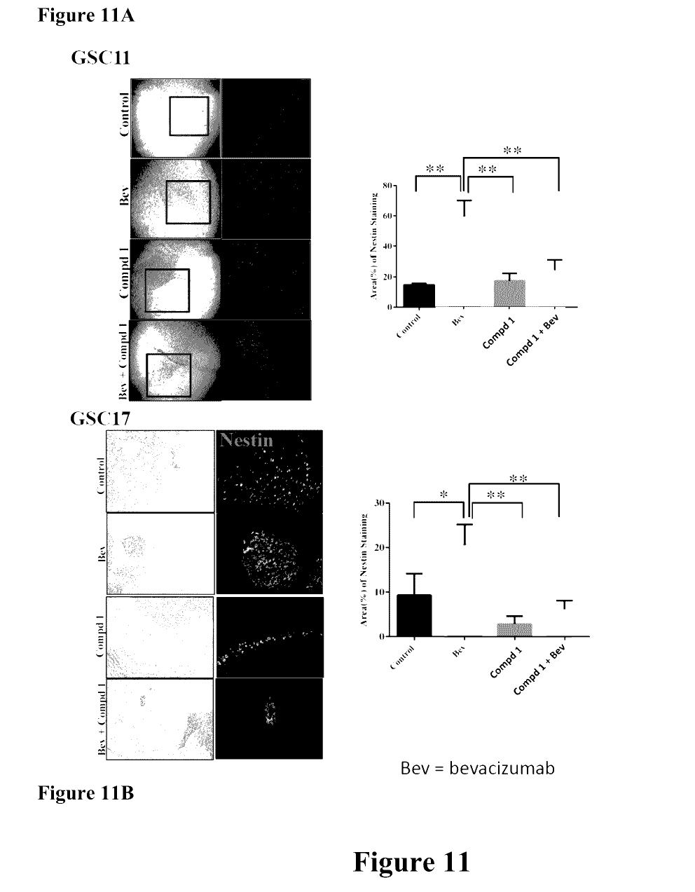

### Compound 1 Blocks Bevacizumab-Mediated Invasiveness and Expression of the Glioma Stem Cell Marker Nestin (FIG. 11A, 11B)

It is well known that tumor cells become more aggressive and more invasive after developing resistance to anti-angiogenic therapy [Piao 2012]. Indeed, in these xenograft mouse models, tumor cells became more invasive in bevacizumab-treated mice compared with the control-treated mice. However, the tumors treated with Compound 1 alone showed very little invasiveness, and Compound 1 combined with bevacizumab dramatically decreased invasiveness compared with bevacizumab alone, as detailed below.

To quantify invasiveness, tissue sections were stained with the glioblastoma stem cell marker nestin and quantified the nestin-positive staining area. In the GSC11 xenograft mouse model, the nestin-positive area was 14.8±0.52% in the control-treated tumors and 59.8±6.0% in the bevacizumab-treated tumors (vs control: p=0.0017). However, the nestin-positive area was only 24.5±3.9% in the tumors treated with Compound 1 combined with bevacizumab (vs control: p=0.0717; vs bevacizumab alone: p=0.0079) and 17.7±2.7% in the tumors treated with Compound 1 alone (FIG. 11A). Similarly, in the GSC17 xenograft mouse model, the nestin-positive area was 9.3±2.8% in the control-treated tumors and 20.6±2.7% in the bevacizumab-treated tumors (vs control: p=0.0423). However, the nestin-positive area was only 6.1±1.1% in the tumors treated with Compound 1 combined with bevacizumab (vs control: p=0.3541; vs bevacizumab alone: p=0.0073) and 2.9±1.0% in the tumors treated with Compound 1 alone (FIG. 11B).

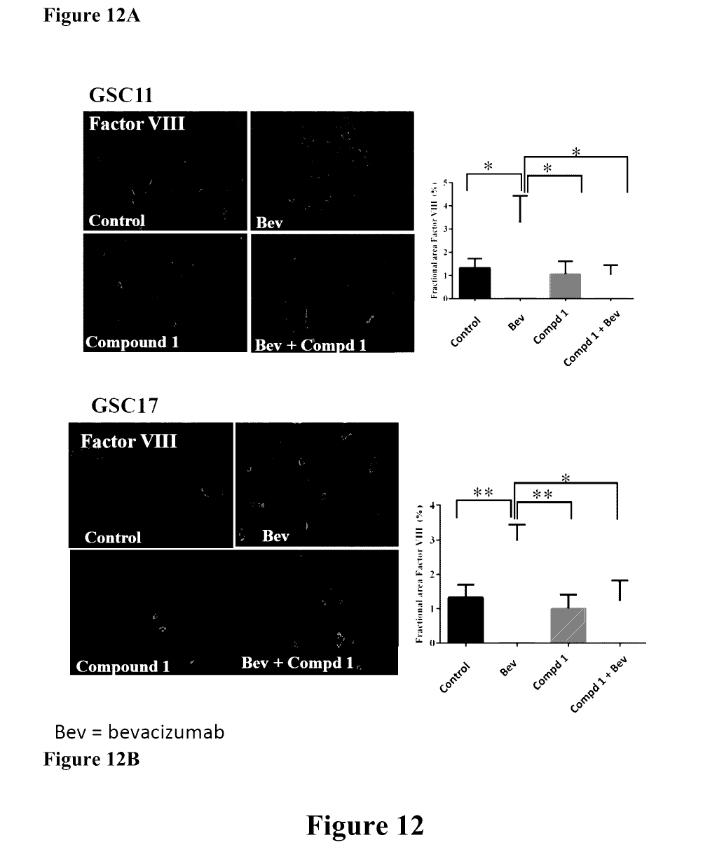

### Compound 1 Blocks Bevacizumab-Mediated Evasive Revascularization (FIG. 12A, B)

Glioblastoma stem cells escape anti-VEGF therapy through revascularization and become more invasive in vivo [Piao 2012]. We performed immunofluorescence staining for the vascular marker Factor VIII and quantified the Factor VIII-positive staining area at the tumor sites in GSC11 and GSC17 xenograft mouse models. In the GSC11 model, the Factor VIII-positive area was 1.3±0.4% in the control-treated tumors and 3.3±1.1% in the bevacizumab-treated tumors (vs control: p=0.0476). However, the Factor VIII-positive area was only 1.03±0.4% in the tumors treated with Compound 1 combined with bevacizumab (vs bevacizumab alone: p=0.0315) and 1.1±0.5% in the tumors treated with Compound 1 alone (vs bevacizumab alone: p=0.0375; (FIG. 12A).

Similarly, in the GSC17 xenograft mouse model, the Factor VIII-positive area was 3.0±0.3% in the tumors treated with bevacizumab (vs control: p=0.0084). However, the Factor VIII-positive area was only 1.2±0.4% in the tumors treated with Compound 1 combined with bevacizumab (vs bevacizumab alone: p=0.0157) and 1.0±0.2% in the tumors treated with Compound 1 alone (vs bevacizumab alone: p=0.0049; (FIG. 12B).

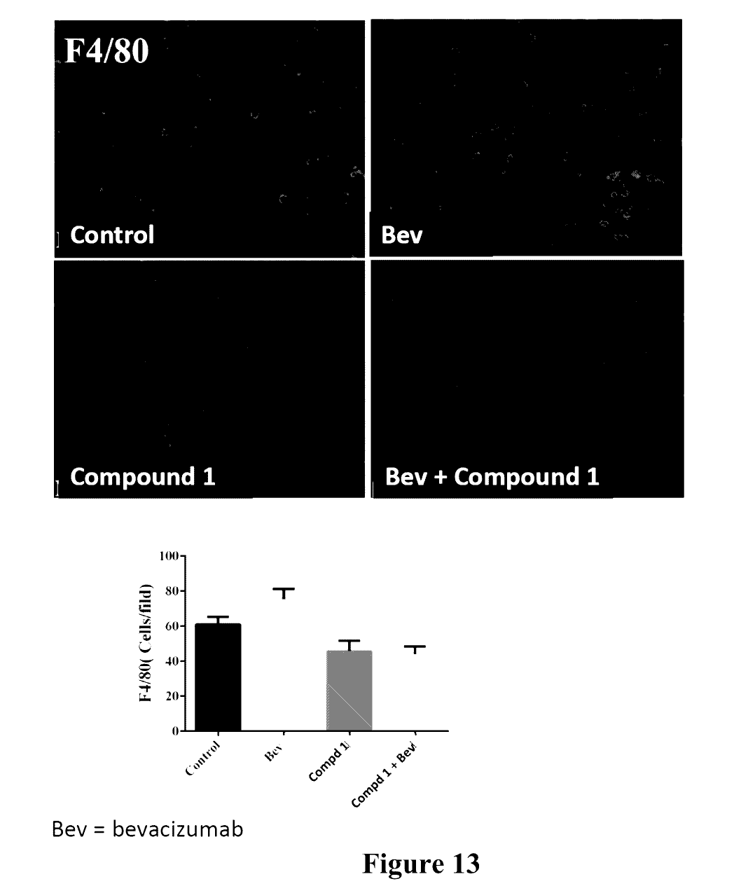

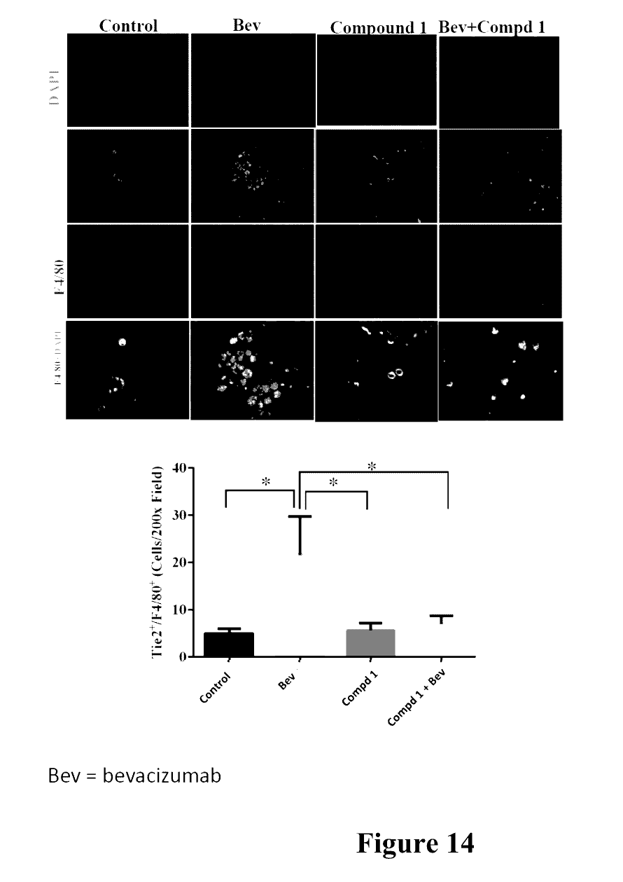

### Compound 1 Blocks Bevacizumab-Mediated Recruitment of Protumoral Macrophages (FIGS. 13-14)

TIE2 is expressed by monocytes observed to accumulate at the invasive edges of malignant gliomas treated with VEGF inhibitors [Gabrusiewicz 2014]. We investigated whether Compound 1 inhibits infiltration of these monocytes into glioblastomas by performing co-immunofluorescence staining for TIE2 and F4/80

FIG. 13 demonstrates that treatment with single agent bevacizumab results in therapy resistance by stimulating the influx of tumor-promoting macrophages (as quantified by F4/80 IHC staining) in the GSC-11 glioma xenograft model. Compound 1 as a single agent therapy does not result in therapy resistance-mediated increases in macrophage infiltration, and also in combination, Compound 1 reverses the effects of bevacizumab in causing such macrophage infiltration into the glioma tumor.

In the GSC-17 glioma xenograft model, bevacizumab treatment increased the tumor infiltration of TIE2-positive/F4/80-positive cells compared to controls (vs control: p=0.0239). However, the infiltration of TIE2-positive/F4/80-positive cells was lower in tumors treated with Compound 1 alone and in those treated with Compound 1 combined with bevacizumab than in tumors treated with bevacizumab alone (p=0.0281 and p=0.0375 respectively; FIG. 14). Notably, Compound 1 in combination with bevacizumab completely ablated bevacizumab-induced increases in TIE2-expressing monocyte recruitment.

### Example 12

### Evaluation of Compound 1 in the PyMT Syngeneic Breast Cancer Model

Compound 1 was evaluated in vivo in the PyMT syngeneic model of mammary carcinoma. This model recapitulates many features of human breast cancer stage and progression and leads to distant lung metastasis that has been demonstrated to involve ANG/TIE2 involvement [Guy 1992; Huang 2011]. Female FVB/NJ mice were implanted in the 4th mammary fat pad with one million cells that had been dissociated from tumor fragments from MMTV-PyMT donor mice. Treatments began on Day 31 when the mean tumor burden in the experiment was 843 mg. Mice (n=10/group) were dosed for three weeks, then, primary tumor and lungs were collected and fixed. Lung sections were stained with H&E at Premier Laboratory. Lung metastases (≧10 cells) were counted manually via microscopy. Data were analyzed via one-way analysis of variance (ANOVA), with post-hoc analysis by the method of Holm-Sidak. For analysis of TIE2-positive macrophages, tumor sections were stained with TIE2 and CD31 antibodies at Premier Laboratory. The density of TIE2-positive cells at the stroma/tumor boundary in each tumor was scored using 0=no staining, 1=low, 2=medium, and 3=high.

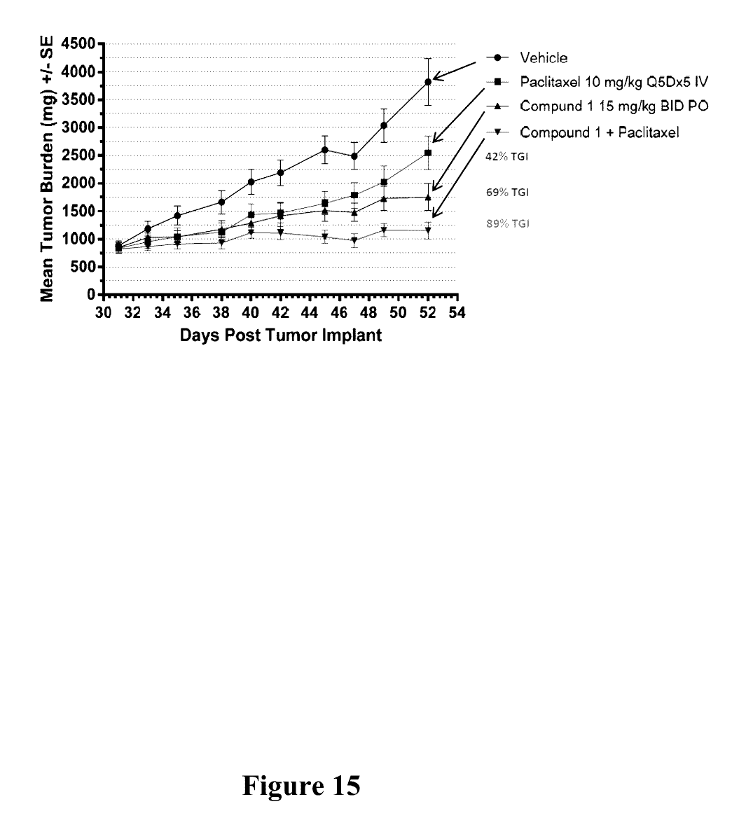

Compound 1 Alone and in Combination with Paclitaxel Inhibits PyMT Mammary Tumor Growth, Reduces Tumoral TIE2⁺ Stromal Cell Density, and Inhibits Lung Metastasis (FIGS. 15-17)

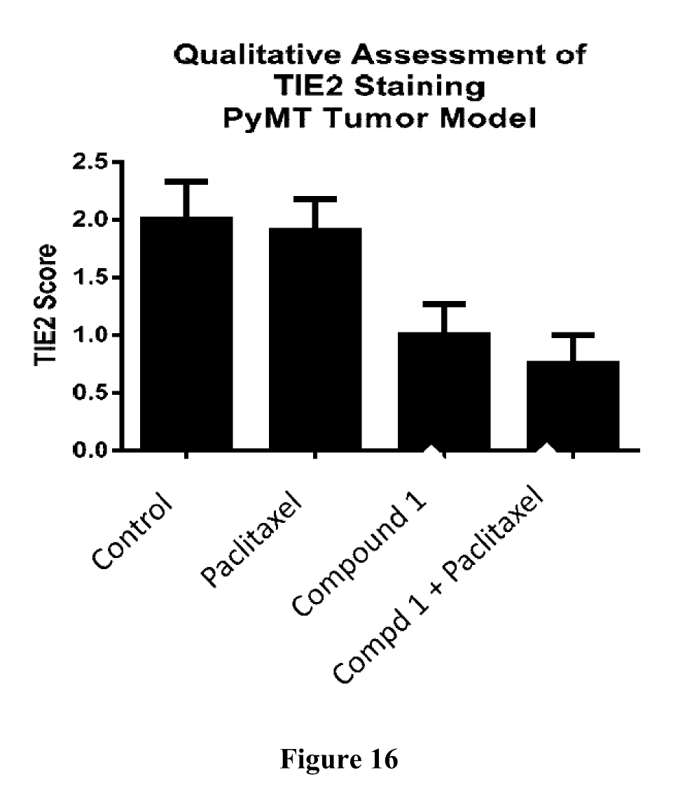

Compound 1 was evaluated in the PyMT syngeneic mammary tumor model. After staging syngeneic mice with implanted PyMT cells in the mammary fat pad and allowing the primary tumors to reach ˜850 mg, cohorts were randomized and treated with vehicle, paclitaxel single agent (10 mg/kg i.v. every 5 days), Compound 1 (15 mg/kg twice daily), or a combination of the two agents. After dosing for three weeks, tumor growth inhibition (TGI) was determined to be 42% for the paclitaxel cohort (p=0.013), 69% for the Compound 1 cohort (p=0.002), and 89% for the combination cohort (p<0.0001) (FIG. 15).

Whereas paclitaxel single agent did not reduce stromal density of TIE2⁺ cells, Compound 1 as a single agent and in combination with paclitaxel reduced the TIE2 score by 50% (p=0.043) and 62% (p=0.015), respectively (FIG. 16).

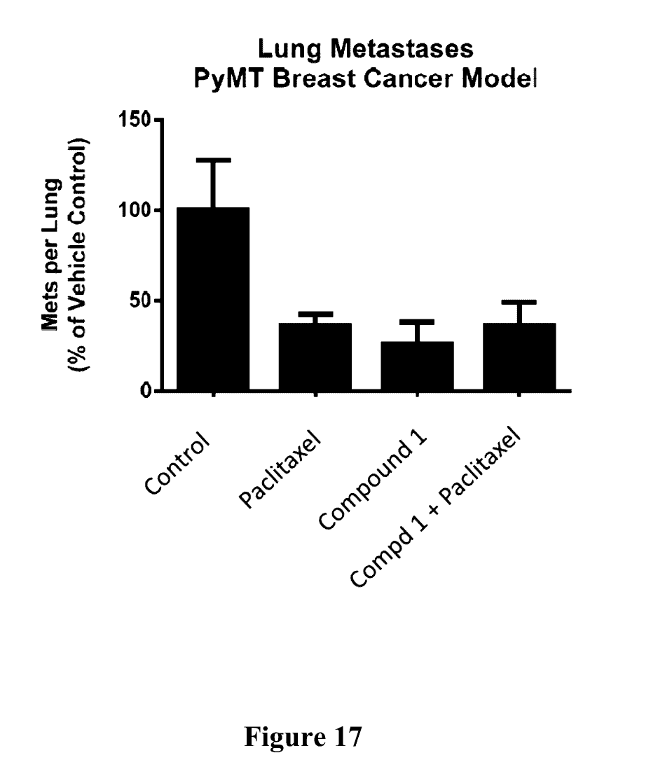

Compound 1 as a single agent reduced lung metastases by 74% (p=0.012), and the paclitaxel and combination cohorts reduced lung metastases similarly by 64% (p=0.020) compared to controls (FIG. 17).

### Example 13

### Evaluation of Compound 1 in Other Tumor Models

B16/F10 metastatic melanoma model. The B16/F10 syngeneic melanoma model is a highly aggressive MET-expressing melanoma tumor noteworthy both for its rapid growth and high rate of pulmonary metastases mediated at least in part by circulating MET⁺/TIE2⁺ proangiogenic monocytes [Peinado 2012]. Mice were implanted subcutaneously in the right lower back on Day 0 with 1×10⁶ luciferase-enabled B16/F10 cells. Treatment began on Day 0. Treatment with Compound 1 (15 mg/kg) resulted in a statistically significant reduction in tumor growth on Day 21 (% T/C=30%; T-C>4.3 days; p<0.05). Compound 1 (15 mg/kg) significantly inhibited the development of pulmonary metastases by 72% in the B16/F10 model: Metastases occurred in 16.6% (2/12) of treated animals on Day 21 (based on ex vivo bioluminescent imaging (BLI) after an intraperitoneal injection of Luciferin) compared to 60% (6/10) in the control animals (p=0.074).

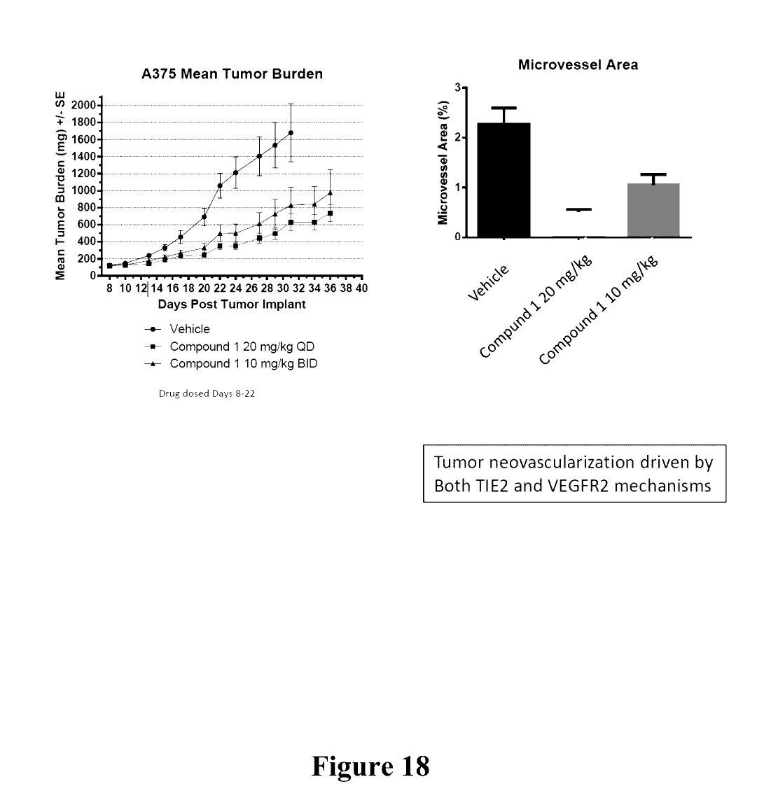

A375 colorectal cancer model (FIG. 18). Compound 1 was evaluated for single-agent efficacy in the A375 BRAF V600E xenograft model (FIG. 18). Though Compound 1 only weakly inhibited BRAF-V600E cell proliferation in vitro, its pan anti-angiogenic inhibitory properties made it a candidate for evaluation on tumor growth mediated by effects on tumor vascularization. Mice were implanted subcutaneously and treatment began on Day 8. Treatments ended on Day 36 after four weeks of treatment. Treatment with Compound 1 (20 mg/kg, orally, QD) produced a statistically significant tumor growth delay of 15.7 days (p<0.001), and a Day 22% T/C of 33% (p<0.05). Treatment with Compound 1 (10 mg/kg, orally, BID) produced a statistically significant median tumor growth delay of >12.4 days (p<0.001) and a Day 22% T/C value of 47% (p<0.05). Inhibition of tumor growth correlated with significant decreases in CD31⁺ microvessel area in tumor sections at the end of study, confirming the effect of Compound 1 in reducing tumor vascularization (FIG. 18).

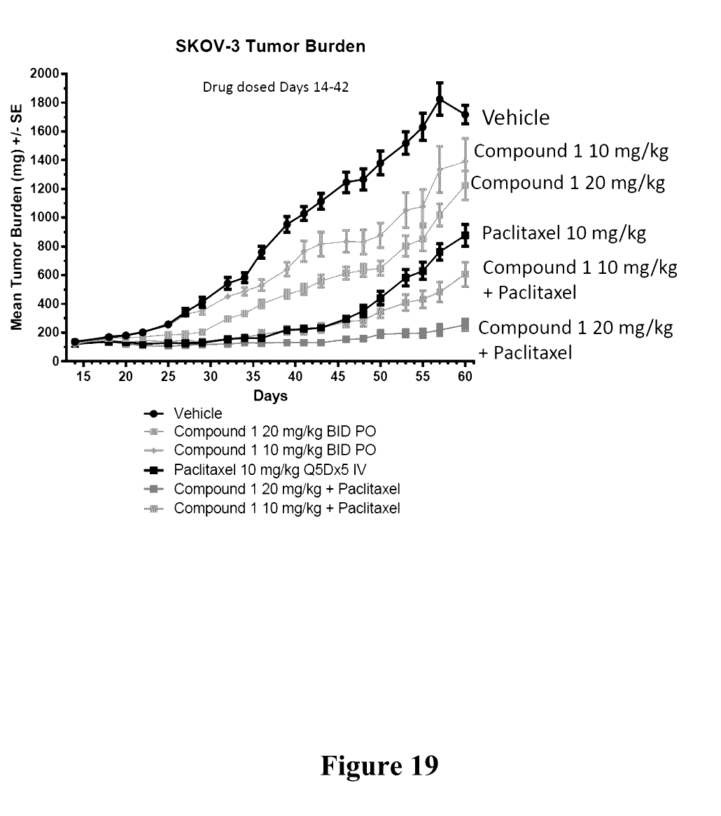

SKOV-3 ovarian cancer model (FIG. 19). Compound 1 was evaluated for efficacy in the SKOV-3 ovarian xenograft model (FIG. 19). Though Compound 1 only weakly inhibited SKOV-3 tumor growth as a single agent, in combination with paclitaxel, Compound 1 significantly reduced tumor growth compared to single agent paclitaxel.

Compound 1 exhibits balanced inhibition of TIE2, MET, and VEGFR2 kinases within a single therapeutic. By inhibiting these kinases with balanced potency, Compound 1 addresses multiple hallmarks of cancer [Hanahan 2011], including cancer cell specific mechanisms of MET-mediated tumor initiation and progression, and tumor microenvironment mechanisms driven by TIE2, MET, and VEGFR2 including angiogenesis, paracrine activation of tumor cells by stromal HGF, invasion, metastasis, and inflammation. The biological data summarized above support the use of Compound 1 to block tumor growth, invasion, and metastasis by blocking key interactions between the tumor cells and the surrounding tumor microenvironment. Additionally, the biological data demonstrate that Compound 1 in combination with anti-tumor agents including anti-VEGF therapy (bevacizumab), chemotherapy (paclitaxel), or other cancer cell targeted therapeutics (dabrafenib) results in superior blockade of tumor growth, invasion, or metastasis compared to single agent anti-VEGF therapy, chemotherapy, or cancer cell targeted therapeutics.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications and this disclosure.

Those skilled in the art will recognize, or be able to ascertain, using no more than routine experimentation, numerous equivalents to the specific embodiments described specifically in this disclosure. Such equivalents are intended to be encompassed in the scope of the following claims.

### REFERENCES

Bergers G, D. Hanahan, Modes of resistance to anti-angiogenic therapy. Nature reviews. Cancer 8, 592-603 (2008).
Brunckhorst M K, Wang H, Lu R, Yu Q. Angiopoietin-4 promotes glioblastoma progression by enhancing tumor cell viability and angiogenesis. Cancer Res. 2010 Sep. 15; 70(18):7283-93.
Coffelt S B, Y. Y. Chen, M. Muthana, A. F. Welford, A. O. Tal, A. Scholz, K. H. Plate, Y. Reiss, C. Murdoch, M. De Palma, C. E. Lewis, Angiopoietin 2 stimulates TIE2-expressing monocytes to suppress T cell activation and to promote regulatory T cell expansion. Journal of immunology 186, 4183-4190 (2011).
Danilkovitch-Miagkova A, Zbar B. Dysregulation of Met receptor tyrosine kinase activity in invasive tumors. J Clin Invest 2002; 109: 863-67.
DeNardo D G, D. J. Brennan, E. Rexhepaj, B. Ruffell, S. L. Shiao, S. F. Madden, W. M. Gallagher, N. Wadhwani, S. D. Keil, S. A. Junaid, H. S. Rugo, E. S. Hwang, K. Jirstrom, B. L. West, L. M. Coussens, Leukocyte complexity predicts breast cancer survival and functionally regulates response to chemotherapy. Cancer Discov 1, 54-67 (2011).
De Palma M, M. A. Venneri, R. Galli, L. S. Sergi, L. S. Politi, M. Sampaolesi, L. Naldini, Tie2 identifies a hematopoietic lineage of proangiogenic monocytes required for tumor vessel formation and a mesenchymal population of pericyte progenitors. Cancer Cell 8, 211-226 (2005).
Ding S, Merkulova-Rainon T, Han Z C, Tobelem G. HGF receptor up-regulation contributes to the angiogenic phenotype of human endothelial cells and promotes angiogenesis in vitro. Blood. 2003; 101:4816-22.
Escamilla J, S. Schokrpur, C. Liu, S. J. Priceman, D. Moughon, Z. Jiang, F. Pouliot, C. Magyar, J. L. Sung, J. Xu, G. Deng, B. L. West, G. Bollag, Y. Fradet, L. Lacombe, M. E. Jung, J. Huang, L. Wu, CSF1 Receptor Targeting in Prostate Cancer Reverses Macrophage-Mediated Resistance to Androgen Blockade Therapy. Cancer research, (2015).
Gabrusiewicz K, D. Liu, N. Cortes-Santiago, M. B. Hossain, C. A. Conrad, K. D. Aldape, G. N. Fuller, F. C. Marini, M. M. Alonso, M. A. Idoate, M. R. Gilbert, J. Fueyo, C. Gomez-Manzano, Anti-vascular endothelial growth factor therapy-induced glioma invasion is associated with accumulation of Tie2-expressing monocytes. Oncotarget 5, 2208-2220 (2014).
Guy C T, Cardiff R D, Muller W J. Induction of mammary tumors by expression of polyomavirus middle T oncogene: a transgenic mouse model for metastatic disease. Mol Cell Biol 1992; 12:954-61.
Hanahan D, Weinberg R A. Hallmarks of Cancer: The Next Generation. Cell 2011; 144:646.
Hashizume H, Falcón B L, Kuroda T, Baluk P, Coxon A, Yu D, et al. Complementary actions of inhibitors of angiopoietin-2 and VEGF on tumor angiogenesis and growth. Cancer Res. 2010 Mar. 15; 70(6):2213-23.
Hata K, Udagawa J, Fujiwaki R, Nakayama K, Otani H, Miyazaki K. Expression of angiopoietin-1, angiopoietin-2, and Tie2 genes in normal ovary with corpus luteum and in ovarian cancer. Oncology. 2002; 62(4):340-8.
Huang H, Lai J Y, Do J, Liu D, Li L, Del Rosario J, et al. Specifically targeting angiopoietin-2 inhibits angiogenesis, Tie2-expressing monocyte infiltration, and tumor growth. Clin Cancer Res. 2011; 17:1001-11.
Ibberson M, S. Bron, N. Guex, E. Faes-van't Hull, A. Ifticene-Treboux, L. Henry, H. A. Lehr, J. F. Delaloye, G. Coukos, I. Xenarios, M. A. Doucey, TIE-2 and VEGFR kinase activities drive immunosuppressive function of TIE-2-expressing monocytes in human breast tumors. Clinical Cancer Research 19, 3439-3449 (2013).
Jahangiri A, De Lay M, Miller L M, Carbonell W S, Hu YL, Lu K, et al. Gene expression profile identifies tyrosine kinase c-Met as a targetable mediator of antiangiogenic therapy resistance. Clin Cancer Res. 2013; 19:1773-83.
Kitajima Y, Ide T, Ohtsuka T, Miyazaki K. Induction of hepatocyte growth factor activator gene expression under hypoxia activates the hepatocyte growth factor/c-Met system via hypoxia inducible factor-1 in pancreatic cancer. Cancer Sci. 2008 July; 99(7): 1341-7.
Lin E Y, J. F. Li, L. Gnatovskiy, Y. Deng, L. Zhu, D. A. Grzesik, H. Qian, X. N. Xue, J. W. Pollard, Macrophages regulate the angiogenic switch in a mouse model of breast cancer. Cancer Res 66, 11238-11246 (2006).
Lu K V, Chang J P, Parachoniak C A, Pandika M M, Aghi M K, Meyronet D, et al. VEGF inhibits tumor cell invasion and mesenchymal transition through a MET/VEGFR2 complex. Cancer Cell 2012; 22:21-35.
Mazzieri R, Pucci F, Moi D, Zonari E, Ranghetti A, Berti A, et al. Targeting the ANG2/TIE2 axis inhibits tumor growth and metastasis by impairing angiogenesis and disabling rebounds of proangiogenic myeloid cells. Cancer Cell. 2011; 19:512-26.
Mitchem J B, D. J. Brennan, B. L. Knolhoff, B. A. Belt, Y. Zhu, D. E. Sanford, L. Belaygorod, D. Carpenter, L. Collins, D. Piwnica-Worms, S. Hewitt, G. M. Udupi, W. M. Gallagher, C. Wegner, B. L. West, A. Wang-Gillam, P. Goedegebuure, D. C. Linehan, D. G. DeNardo, Targeting tumor-infiltrating macrophages decreases tumor-initiating cells, relieves immunosuppression, and improves chemotherapeutic responses. Cancer research 73, 1128-1141 (2013).
Nakasone E S, H. A. Askautrud, T. Kees, J. H. Park, V. Plaks, A. J. Ewald, M. Fein, M. G. Rasch, Y. X. Tan, J. Qiu, J. Park, P. Sinha, M. J. Bissell, E. Frengen, Z. Werb, M. Egeblad, Imaging tumor-stroma interactions during chemotherapy reveals contributions of the microenvironment to resistance. Cancer Cell 21, 488-503 (2012).
Peinado H, Ale\check{c}ković M, Lavotshkin S, Matei I, Costa-Silva B, Moreno-Bueno G, et al. Melanoma exosomes educate bone marrow progenitor cells toward a pro-metastatic phenotype through MET. Nat Med. 2012; 18:883-91.
Piao Y, Liang J, Holmes L, Henry V, Sulman E, de Groot J F. Acquired resistance to anti-VEGF therapy in glioblastoma is associated with a mesenchymal transition. Clin Cancer Res. 2013; 19:4392-403.
Piao Y, Liang J, Holmes L, et al. Glioblastoma resistance to anti-VEGF therapy is associated with myeloid cell infiltration, stem cell accumulation, and a mesenchymal phenotype. Neuro Oncol. 2012; 14(11):1379-1392.
Pucci F, M. A. Venneri, D. Biziato, A. Nonis, D. Moi, A. Sica, C. Di Serio, L. Naldini, M. De Palma, A distinguishing gene signature shared by tumor-infiltrating Tie2-expressing monocytes, blood “resident” monocytes, and embryonic macrophages suggests common functions and developmental relationships. Blood 114, 901-914 (2009).
Rigamonti N, Kadioglu E, Keklikoglou I, Wyser Rmili C, et al. Role of angiopoietin-2 in adaptive tumor resistance to VEGF signaling blockade. Cell Reports 2014; 8:696-706.
Robinson B D, G. L. Sica, Y. F. Liu, T. E. Rohan, F. B. Gertler, J. S. Condeelis, J. G. Jones, Tumor microenvironment of metastasis in human breast carcinoma: a potential prognostic marker linked to hematogenous dissemination. Clinical Cancer Research 15, 2433-2441 (2009).
Rohan T E, X. Xue, H. M. Lin, T. M. D'Alfonso, P. S. Ginter, M. H. Oktay, B. D. Robinson, M. Ginsberg, F. B. Gertler, A. G. Glass, J. A. Sparano, J. S. Condeelis, J. G. Jones, Tumor microenvironment of metastasis and risk of distant metastasis of breast cancer. Journal of the National Cancer Institute 106, (2014).
Ruffell B, L. M. Coussens, Macrophages and Therapeutic Resistance in Cancer. Cancer cell 27, 462-472 (2015).
Sennino B, Ishiguro-Oonuma T, Wei Y, Naylor R M, Williamson C W, Bhagwandin V, et al. Suppression of Tumor Invasion and Metastasis by Concurrent Inhibition of c-Met and VEGF Signaling in Pancreatic Neuroendocrine Tumors. Cancer Discovery 2012; 2: OF1-OF18.
Sennino B, Ishiguro-Oonuma T, Schriver B J, Christensen J G, McDonald D M. Inhibition of c-Met Reduces Lymphatic Metastasis in RIP-Tag2 Transgenic Mice. Cancer Res 2013; 73:3692-3703.
Schindler, T, Bornmann, W, Pellicena, P, Miller, W T, Clarkson, B, Kuriyan, J. Structural mechanism for STI-571 inhibition of Abelson tyrosine kinase. Science 2000; 289: 1938-42.
Schulz P, Fischer C, Detjen K M, Rieke S, Hilfenhaus G, von Marschall Z, et al. Angiopoietin-2 drives lymphatic metastasis of pancreatic cancer. FASEB J. 2011; 25:3325-35.
Shree T, O. C. Olson, B. T. Elie, J. C. Kester, A. L. Garfall, K. Simpson, K. M. Bell-McGuinn, E. C. Zabor, E. Brogi, J. A. Joyce, Macrophages and cathepsin proteases blunt chemotherapeutic response in breast cancer. Genes Dev 25, 2465-2479 (2011).
Strachan D C, B. Ruffell, Y. Oei, M. J. Bissell, L. M. Coussens, N. Pryer, D. Daniel, CSF1R inhibition delays cervical and mammary tumor growth in murine models by attenuating the turnover of tumor-associated macrophages and enhancing infiltration by CD8 T cells. Oncoimmunology 2, e26968 (2013).
Straussman R, Morikawa T, Shee K, Barzily-Rokni M, Qian Z R, Du J, Davis A, et al. Tumour micro-environment elicits innate resistance to RAF inhibitors through HGF secretion. Nature. 2012; 487:500-4.
Van Belle E, Witzenbichler B, Chen D, Silver M, Chang L, Schwall R, et al. Potentiated Angiogenic Effect of Scatter Factor/Hepatocyte Growth Factor via Induction of Vascular Endothelial Growth Factor. Circulation 1998; 97:381-390.
Wang W, Li Q, Yamada T, Matsumoto K, Matsumoto I, Oda M, et al. Crosstalk to stromal fibroblasts induces resistance of lung cancer to epidermal growth factor receptor tyrosine kinase inhibitors. Clin Cancer Res. 2009; 15:6630-8.
Welford A F, D. Biziato, S. B. Coffelt, S. Nucera, M. Fisher, F. Pucci, C. Di Serio, L. Naldini, M. De Palma, G. M. Tozer, C. E. Lewis, TIE2-expressing macrophages limit the therapeutic efficacy of the vascular-disrupting agent combretastatin A4 phosphate in mice. The Journal of clinical investigation 121, 1969-1973 (2011).
Wilson T R, Fridlyand J, Yan Y, Penuel E, Burton L, Chan E, Peng J, et al. Widespread potential for growth-factor-driven resistance to anticancer kinase inhibitors. Nature. 2012; 487:505-9.
Wyckoff J, W. Wang, E. Y. Lin, Y. Wang, F. Pixley, E. R. Stanley, T. Graf, J. W. Pollard, J. Segall, J. Condeelis, A paracrine loop between tumor cells and macrophages is required for tumor cell migration in mammary tumors. Cancer Res 64, 7022-7029 (2004).
Wyckoff J B, Y. Wang, E. Y. Lin, J. F. Li, S. Goswami, E. R. Stanley, J. E. Segall, J. W. Pollard, J. Condeelis, Direct visualization of macrophage-assisted tumor cell intravasation in mammary tumors. Cancer Res 67, 2649-2656 (2007).
Xin X, Yang S, Ingle G, Zlot C, Rangell L, Kowalski J, et al. Hepatocyte growth factor enhances vascular endothelial growth factor-induced angiogenesis in vitro and in vivo. Am J Pathol. 2001 March; 158(3):1111-20.
Yano S, Yamada T, Takeuchi S, Tachibana K, Minami Y, Yatabe Y, et al. Hepatocyte growth factor expression in EGFR mutant lung cancer with intrinsic and acquired resistance to tyrosine kinase inhibitors in a Japanese cohort. J Thorac Oncol. 2011; 12:2011-7.

1. A method for treating solid tumors, gastrointestinal stromal tumors, glioblastoma, melanoma, ovarian cancer, breast cancer, renal cancer, hepatic cancer, cervical carcinoma, non small cell lung cancer, mesothelioma, or colon cancer comprising administering to a patient in need thereof an effective amount of N-(4-(2-(cyclopropanecarboxamido)pyridin-4-yloxy)-2,5-difluorophenyl)-N′-(4-fluorophenyl)cyclopropane-1,1-dicarboxamide, or a pharmaceutically acceptable salt thereof.

2. The method of claim 1, wherein the N-(4-(2-(cyclopropanecarboxamido)pyridin-4-yloxy)-2,5-difluorophenyl)-N′-(4-fluorophenyl)cyclopropane-1,1-dicarboxamide or pharmaceutically acceptable salt thereof inhibits tumor cell interactions with the microenvironment.

3. The method of claim 1, wherein the N-(4-(2-(cyclopropanecarboxamido)pyridin-4-yloxy)-2,5-difluorophenyl)-N′-(4-fluorophenyl)cyclopropane-1,1-dicarboxamide or pharmaceutically acceptable salt thereof inhibits angiopoietin (ANG) signaling through TIE2 kinase in the tumor microenvironment.

4. The method of claim 1, wherein the N-(4-(2-(cyclopropanecarboxamido)pyridin-4-yloxy)-2,5-difluorophenyl)-N′-(4-fluorophenyl)cyclopropane-1,1-dicarboxamide or pharmaceutically acceptable salt thereof inhibits three microenvironment (re)vascularization and drug resistance pathways (ANG, HGF, VEGF), that signal through receptor tyrosine kinases (TIE2, MET, VEGFR2, respectively).

5. The method of claim 1, wherein N-(4-(2-(cyclopropanecarboxamido)pyridin-4-yloxy)-2,5-difluorophenyl)-N′-(4-fluorophenyl)cyclopropane-1,1-dicarboxamide is administered in combination with another chemotherapeutic agent.

6. The method of claim 5, wherein the chemotherapeutic agent is an anti-tubulin agent.

7. The method of claim 6, wherein the anti-tubulin agent is taken from paclitaxel, docetaxel, abraxane, or eribulin.

8. The method of claim 1, wherein N-(4-(2-(cyclopropanecarboxamido)pyridin-4-yloxy)-2,5-difluorophenyl)-N′-(4-fluorophenyl)cyclopropane-1,1-dicarboxamide is administered in combination with another anti-cancer targeted therapeutic agent.

9. The method of claim 8, wherein the other targeted therapeutic agent is a kinase inhibitor.

10. The method of claim 9, wherein the other targeted therapeutic agent is a BRAF kinase inhibitor.

11. The method of claim 10, wherein the BRAF inhibitor is dabrafenib or vemurafenib.

12. The method of claim 1, wherein N-(4-(2-(cyclopropanecarboxamido)pyridin-4-yloxy)-2,5-difluorophenyl)-N′-(4-fluorophenyl)cyclopropane-1,1-dicarboxamide is administered in combination with another immunotherapy agent.

13. The method of claim 12, wherein the other immunotherapy agent is an anti-CTLA-4 agent, an anti-PD agent, an anti-PDL agent, or an IDO inhibitor.

14. The method of claim 13, wherein the other immunotherapy agent is ipilimumab.

15. The method of claim 13, wherein the other immunotherapy agent is pembrolizumab or nivolumab.

16. The method of claim 13, wherein the other immunotherapy agent is atezolizumab avelumab, or MEDI4736.

17. The method of claim 13, wherein the other immunotherapy agent is indoximod, INCB024360, or epacadostat.

18. The method of claim 1, wherein N-(4-(2-(cyclopropanecarboxamido)pyridin-4-yloxy)-2,5-difluorophenyl)-N′-(4-fluorophenyl)cyclopropane-1,1-dicarboxamide is administered in combination with another anti-angiogenic agent.

19. The method of claim 18, wherein the other anti-angiogenic agent is bevacizumab.

20. The method of claim 1, wherein the effective amount of N-(4-(2-(cyclopropanecarboxamido)pyridin-4-yloxy)-2,5-difluorophenyl)-N′-(4-fluorophenyl)cyclopropane-1,1-dicarboxamide, or pharmaceutically acceptable salt thereof is administered to the subject orally.


SEQUENCE LISTING (3 sequences)

SEQ ID NO: 1 - protein, 354 aa, Homo sapiens
QLKRANVQRRMAQAFQNVREEPAVQFNSGTLALNRKVKNNPDPTIYPVLDWNDIKFQDVIGEGNFGQVLKARIKKDGLRMDAAIKRMKEYASKDDHRDFAGELEVLCKLGHHPNIINLLGACEHRGYLYLAIEYAPHGNLLDFLRKSRVLETDPAFAIANSTASTLSSQQLLHFAADVARGMDYLSQKQFIHRDLAARNILVGENYVAKIADFGLSRGQEVYVKKTMGRLPVRWMAIESLNYSVYTTNSDVWSYGVLLWEIVSLGGTPYCGMTCAELYEKLPQGYRLEKPLNCDDEVYDLMRQCWREKPYERPSFAQILVSLNRMLEERKTYVNTTLYEKFTYAGIDCSAEEAA

SEQ ID NO: 2 - protein, 311 aa, Homo sapiens
TVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVK

SEQ ID NO: 3 - protein, 315 aa, Homo sapiens
DPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKVAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQD